(19) United States
(12) Patent Application Publication
(10) Pub. No.: US 2021/0202090 A1
(43) Pub. Date: Jul. 1, 2021

(54) AUTOMATED HEALTH CONDITION SCORING IN TELEHEALTH ENCOUNTERS

(71) Applicant: TELADOC HEALTH, INC., Purchase, NY (US)

(72) Inventors: John O'Donovan, Goleta, CA (US); Pushkar Shukla, Chicago, IL (US); Paul C. McElroy, Goleta, CA (US); Sushil Bharati, Goleta, CA (US); Marco Pinter, Santa Barbara, CA (US)

(21) Appl. No.: 16/949,370

(22) Filed: Oct. 27, 2020

Related U.S. Application Data
(60) Provisional application No. 62/953,858, filed on Dec. 26, 2019.

(51) Int. Cl. G16H 50/20; G16H 50/30; G16H 40/67; G16H 15/00; G06N 3/08; G10L 15/26; G10L 15/22; G10L 25/66; G06K 9/00; A61B 5/00; A61B 5/11

(52) U.S. Cl. CPC G16H 50/20; G16H 10/60; G16H 40/67; G16H 15/00; G06N 3/08; G10L 15/26; G10L 15/22; G10L 25/66; G06K 9/00711; G06K 9/00268; G06K 9/00355; A61B 5/7267; A61B 5/1124; A61B 5/4803; A61B 5/4064; A61B 5/7282; G16H 50/30

(57) ABSTRACT

A system for automated health condition scoring includes at least one communication interface to receive an audio stream and a video stream from an endpoint in proximity to a patient, at least two different artificial intelligence (“Al”) detectors to respectively process one or both of the audio stream and the video stream using machine learning to automatically determine at least two respective likelihoods of the patient having a health condition, an Al scorer to combine the at least two respective likelihoods of the health condition using machine learning to automatically determine a health condition score representing an overall likelihood of the patient having the health condition, and a display interface that displays an indication of the health condition score to a physician.

### CROSS-REFERENCE TO RELATED APPLICATIONS

This application claims the benefit of U.S. Provisional Application No. 62/953,858, filed Dec. 26, 2019, for Al SENSORS FOR STROKE ASSESSMENT IN TELEHEALTH, which is hereby incorporated by reference in its entirety.

### TECHNICAL FIELD

The present disclosure pertains to telehealth systems and more specifically to automated health condition scoring in telehealth encounters.

### BACKGROUND

In the course of examining a patient, a physician relies on a variety of audible and visual cues to make a diagnosis. However, the physician can typically only focus on one symptom at a time. Certain medical conditions present with a number of different symptoms, some of which can be subtle and difficult to detect, particularly in a short time frame and/or under stressful conditions. The difficulty is exacerbated in the context of telehealth where the physician is examining the patient remotely.

Acute cerebral infarction, commonly known as “stroke”, is a restriction of blood flow to the brain that is frequently caused by arterial clots. FAST is an acronym used as a mnemonic to help detect and enhance responsiveness to the needs of a person having a stroke. The acronym stands for Facial drooping, Arm weakness, Speech difficulties, and Time to call emergency services. The first three letters of the acronym correspond to three of the key indicators of a stroke.

Facial drooping, for instance, relates to a section of the face, usually only on one side, that is drooping relative to the other side. Ataxia, or impaired coordination or limb weakness, often includes the inability to raise one's arm fully or maintain one's arm outstretched arm without motion for a period of time. Dysarthria includes various difficulties in producing or understanding speech. Neurologists evaluate a potential stroke victim in each of the foregoing areas, among others.

Since neurologists with expertise in diagnosing and treating stroke are a scarce resource, patients are sometimes treated by a remote neurologist who interviews and examines the patient via a video connection. However, the video connection puts a barrier between the neurologist and the patient, making it easier to miss, for example, subtle degrees of facial asymmetry. The progression of asymmetry during a consultation (or longer duration) is a key indicator of stroke severity. However, such progression may be hard to detect by a neurologist, even when meeting with the patient in person, much less over a video connection.

### SUMMARY

A system for automated health condition scoring may include at least one communication interface to receive an audio stream and a video stream from an endpoint in proximity to a patient. The system may further include at least two different artificial intelligence (“Al”) detectors to respectively process one or both of the audio stream and the video stream using machine learning to automatically determine at least two respective likelihoods of the patient having a health condition.

In one embodiment, the system further includes an Al scorer to combine the at least two respective likelihoods of the health condition using machine learning to automatically determine a health condition score representing an overall likelihood of the patient having the health condition. In some embodiments, the Al scorer may assign a separate weight to each of the at least two respective likelihoods of the health condition in determining the health condition score. After the health condition score is determined, a display interface may then display an indication of the health condition score to a physician.

The system may also include a speech-to-text unit to convert the audio stream into text that is combined by the Al scorer with the at least two respective likelihoods of the health condition using machine learning to automatically determine the overall likelihood of the patient having the health condition.

The Al scorer may be further configured to receive diagnostic data from a medical monitoring device in proximity to the patient. In such an embodiment, the Al scorer is configured to combine the diagnostic data with the at least two respective likelihoods of the health condition using machine learning to automatically determine the overall likelihood of the patient having the health condition.

In one embodiment, the health condition is a stroke, and the at least two different Al detectors are selected from a group consisting of a facial droop detector, an ataxia detector, and slurred speech detector. In some embodiments, the at least two different Al detectors comprise three Al detectors including a facial droop detector, a limb weakness detector, and a slurred speech detector.

The asymmetry detector may process the video stream to automatically determine a first stroke likelihood based on a measurement of facial droop. Concurrently or contemporaneously with the asymmetry detector, the ataxia detector may process the video stream to automatically determine a second stroke likelihood based on a measurement of limb weakness. Concurrently or contemporaneously with the asymmetry detector and/or the ataxia detector, the dysarthria detector may process the audio stream to automatically determine a third stroke likelihood based on a measurement of slurred speech.

After the first, second, and third stroke likelihoods are determined, a stroke scorer may automatically determine a stroke score for the patient based on a combination of the first, second, and third stroke likelihoods. The display interface may then display an indication of the stroke score to a physician.

The stroke scorer may assign a separate weight to each of the first, second, and third stroke likelihoods in calculating the stroke score, which may be performed, for example, by a machine learning system, such as a deep learning neural network. In one embodiment, a feedback process may provide for updating the machine learning system based on physician feedback.

The stroke score may include one or more of a probability, percentage chance or confidence level of whether the patient has experienced, or is experiencing, a stroke. The stroke scorer may compare the first, second, and third stroke likelihoods with respective thresholds in calculating the stroke score. In some embodiments, the stroke score includes the first, second, and third stroke likelihoods and the respective thresholds. Alternatively, or in addition, the stroke score may include a binary indication of whether or not the patient has experienced, or is experiencing, a stroke based on the respective thresholds.

In one embodiment, the video stream includes one or more video frames showing at least eyes and lips of the patient, and the asymmetry detector includes a facial landmark detector to automatically identify a set of facial keypoints in at least one of the one or more video frames, the facial keypoints including at least a point on each eye of the patient and at least one point on opposite sides of the patient's lips. The facial keypoint detector may include or make use of a machine learning system in automatically identifying the set of facial keypoints, which may include a deep learning neural network.

The asymmetry detector may further include a facial droop detector, in communication with the facial landmark detector, which automatically calculates a degree of facial droop by calculating a first line between each eye point; calculating a second line between each lip point; and calculating an angle between the first line and the second line. Thereafter, an asymmetry scorer may automatically determine the first stroke likelihood based on the calculated angle.

In one embodiment, the video stream includes one or more video frames showing a limb of the patient. The ataxia detector may include a pose estimator to automatically identify body keypoints in the one or more video frames. The body keypoints may include, for example, locations of joints on the limb of the patient.

The ataxia detector may further include a limb velocity detector to use the body keypoints to determine a movement velocity of the limb over a time interval in which the patient is instructed to keep the limb motionless. In one embodiment, the limb velocity detector may determine the movement velocity of the limb by calculating a sum of movement velocities for each joint of the limb. A limb weakness scorer may then calculates the second stroke likelihood as a function of the movement velocity of the limb over the time interval. In one embodiment, one or more of the pose estimator and the limb weakness scorer comprise or access a deep learning neural network.

In one embodiment, the time interval for measuring limb velocity is defined by physician input. In another embodiment, the time interval for measuring limb velocity is automatically determined at least in part based on a text transcription of audio communication between the patient and the physician. In some embodiments, the time interval for measuring limb velocity is automatically determined at least in part based on movement of the limb detected by the pose estimator.

The dysarthria detector may include an audio processor to generate a set of audio coefficients from the audio stream and a slurred speech scorer to determine third stroke likelihood based on the audio coefficients. In one embodiment, the coefficients comprise Mel-Frequency Cepstral Coefficients (MFCCs).

The slurred speech scorer may determine the third stroke likelihood by comparing a first set of audio coefficients produced while the patient reads or repeats a pre-defined text with a second set of audio coefficients produced by a reference sample for the pre-defined text. In one embodiment, the slurred speech scorer determines the third stroke likelihood based on the first and second sets of audio coefficients and one or more thresholds. In various embodiments, the slurred speech scorer comprises or accesses a deep learning neural network.

In various embodiments, the asymmetry detector, dysarthria detector, and stroke scorer continuously process the respective audio and video streams to provide a series of real-time stroke scores that are displayed by the display interface.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only example embodiments of the disclosure and are not therefore to be considered to be limiting of its scope, the principles herein are described and explained with additional specificity and detail through the use of the accompanying drawings in which:

FIG. 1 is a schematic diagram of a telehealth system according to one embodiment of the present disclosure.

FIG. 2 is a schematic diagram of a system for automated stroke scoring in a telehealth consultation according to one embodiment of the present disclosure.

FIG. 3A illustrates a process of facial landmark detection and facial droop detection according to one embodiment of the present disclosure.

FIGS. 3B and 3C are graphs of measured facial droop over a time sequence of sampled video frames.

FIG. 4 is a schematic diagram of a stroke scorer according to one embodiment of the present disclosure.

FIG. 5 is a flowchart of a method for automated stroke scoring in a telehealth consultation according to one embodiment of the present disclosure.

FIG. 6 is a schematic diagram of a system for automated stroke scoring in a telehealth consultation according to one embodiment of the present disclosure.

FIG. 7 is a schematic diagram showing additional details of the asymmetry detector and ataxia detector according to one embodiment of the present disclosure.

FIG. 8A through 8D illustrate a process of limb velocity detection according to one embodiment of the present disclosure.

FIG. 9 is a flowchart of a method for determining a measurement of limb weakness according to one embodiment of the present disclosure.

FIG. 10 is a schematic diagram showing additional details of a dysarthria detector according to one embodiment of the present disclosure.

FIG. 11 is a flowchart of a method for determining a measurement of slurred speech according to one embodiment of the present disclosure.

FIG. 12 is a flowchart of a method for determining an overall stoke score based on multiple inputs according to one embodiment of the present disclosure.

FIG. 13 illustrates a user interface for a physician according to one embodiment of the present disclosure.

FIG. 14 is a schematic diagram of a system for automated heath condition scoring in a telehealth consultation according to one embodiment of the present disclosure.

FIG. 15 is a flowchart of a method for determining an overall health condition score based on multiple inputs according to one embodiment of the present disclosure.

FIG. 16 depicts an example computing system that may implement various systems and methods according to embodiments of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

It should be understood at the outset that although illustrative implementations of one or more embodiments are illustrated below, the disclosed apparatus and methods may be implemented using any number of techniques. The disclosure should in no way be limited to the illustrative implementations, drawings, and techniques illustrated herein, but may be modified within the scope of the appended claims along with their full scope of equivalents.

A typical telehealth encounter may involve a patient and one or more remotely located physicians or healthcare providers. Devices located in the vicinity of the patient and the providers allow the patients and providers to communicate with each other using, for example, two-way audio and/or video conferencing.

A telepresence device may take the form of a desktop, laptop, tablet, smart phone, or any computing device equipped with hardware and software configured to capture, reproduce, transmit, and receive audio and/or video to or from another telepresence device across a communication network. Telepresence devices may also take the form of telepresence robots, carts, and/or other devices such as those marketed by InTouch Technologies, Inc. of Santa Barbara, California, under the names INTOUCH VITA, INTOUCH LITE, INTOUCH VANTAGE, INTOUCH VICI, INTOUCH VIEWPOINT, INTOUCH XPRESS, and INTOUCH XPRESS CART. The physician telepresence device and the patient telepresence device may mediate an encounter, thus providing high-quality audio capture on both the provider-side and the patient-side of the interaction.

Furthermore, unlike an in-person encounter where a smart phone may be placed on the table and an application started, a telehealth-based system can intelligently tie into a much larger context around the live encounter. The telehealth system may include a server or cloud infrastructure that provides the remote provider with clinical documentation tools and/or access to the electronic medical record (“EMR”) and medical imaging systems (e.g., such as a “picture archiving and communication system,” or “PACS,” and the like) within any number of hospitals, hospital networks, other care facilities, or any other type of medical information system. In this environment, the software may have access to the name or identification of the patient being examined as well as access to their EMR. The software may also have access to, for example, notes from hospital staff.

In one example, a physician uses a clinical documentation tool within a telehealth software application on a laptop to review a patient record. The physician can click a “connect” button in the telehealth software that connects the physician telepresence device to a telepresence device in the vicinity of the patient. In one example, the patient-side telepresence device may be a mobile telepresence robot with autonomous navigation capability located in a hospital, such as an INTOUCH VITA. The patient-side telepresence may automatically navigate to the patient bedside, and the telehealth software can launch a live audio and/or video conferencing session between the physician laptop and the patient-side telepresence device such as disclosed in U.S. Pub. No. 2005/02044381 and hereby incorporated by reference in its entirety.

In addition to the live video, the telehealth software can display a transcription box. Everything the physician or patient says can appear in the transcription box and may be converted to text. In some examples, the text may be presented as a scrolling marquee or an otherwise streaming text.

Transcription may begin immediately upon commencement of the session. The physician interface may display a clinical documentation tool, including a stroke workflow (e.g., with a NIHSS, or National Institutes of Health Stroke Scale, score, a tPA, or tissue plasminogen activator, calculator, and the like) such as disclosed in U.S. Pub. No. 2009/0259339 and hereby incorporated by reference in its entirety.

Upon completion of the live encounter with the patient, the physician can end the audio and/or video session. The video window closes and, in the case of a robotic patient-side endpoint, the patient-side telepresence device may navigate back to its dock. The physician-side interface may display a patient record (e.g., within a clinical documentation tool). In some examples, physician notes, such as a Subjective, Objective, Assessment, and Plan (SOAP) note may be displayed next to the patient record, as disclosed in U.S. Pub. No. 2018/0308565, which is hereby incorporated by reference in its entirety.

As previously discussed, one type of telehealth encounter may involve a potential stroke victim and a remote neurologist, since neurologists with expertise to diagnose and treat stroke are a scarce resource. However, the video connection puts a barrier between the neurologist and the patient, making it easier to miss, for example, subtle signs of facial asymmetry or droop. The progression of asymmetry during a consult (or longer duration) is a key indicator of stroke severity. However, such progression may be difficult to detect by a neurologist, even when meeting with the patient in person, much less over a video connection.

The following disclosure provides techniques for automated stroke scoring including automated detection of facial asymmetry in telehealth encounters, which improves over conventional techniques in which the neurologist is limited to seeing and/or conversing with the patient over an audio/video connection. The techniques disclosed herein may also improve diagnostic accuracy of an in-person examination and could be used to supplement the information available to a neurologist via augmented reality (AR).

In one embodiment, the disclosed techniques may employ artificial intelligence (Al) using, for example, a deep learning neural network, in order to detect facial asymmetries of a patient consistent with stroke. The neural network can be a Recurrent Neural Network (RNN) built on the CaFE framework from UC Berkeley. The network may be embodied in a software module that executes on one or more servers coupled to the network in the telehealth system. Alternatively, the module may execute on a patient telepresence device or a physician telepresence device.

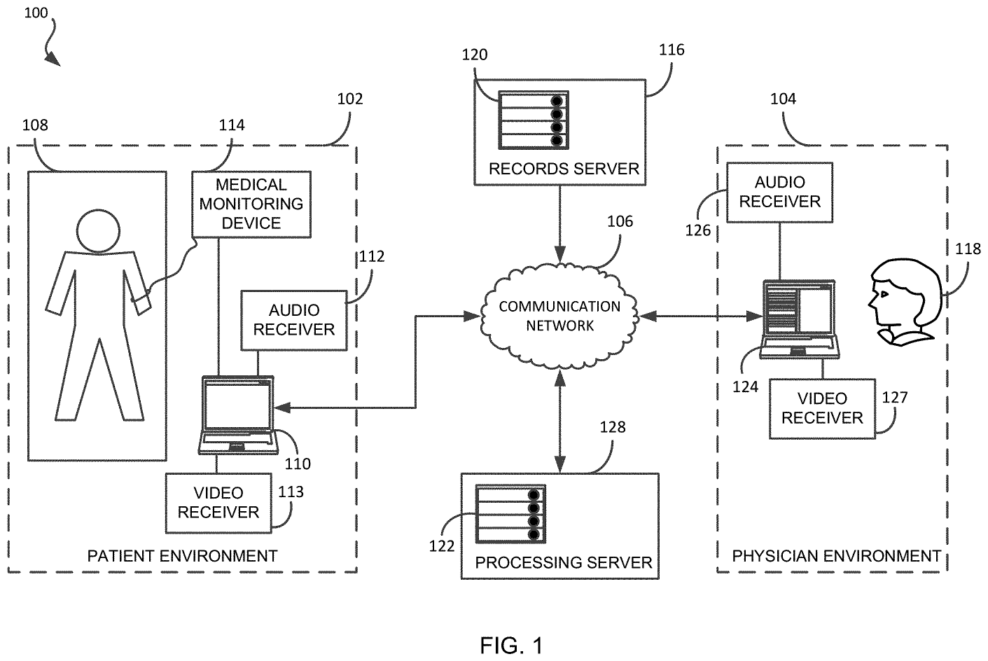

FIG. 1 is a schematic diagram of a telehealth system 100, in which a patient 108 is in a patient environment 102 and a physician 118 in a physician environment 104. In other embodiments, the patient 108 and physician 118 may be in the same environment and/or in close physical proximity, as described more fully hereafter.

The physician 118 and patient 108 may be located in different places and communicate with each other over a communication network 106, which may include one or more Internet linkages, Local Area Networks (“LANs”), mobile networks, proprietary hospital networks, and the like.

In one embodiment, the patient 108 and the physician 118 interact via a patient endpoint 110 in the patient environment 102 and a physician endpoint 124 in the physician environment 104. While depicted in FIG. 1 as computer terminals, it will be understood by a person having ordinary skill in the art that either or both of the patient endpoint 110 and the physician endpoint 124 can be a desktop computer, a mobile phone, a remotely operated robot (i.e., robotic endpoint), a laptop computer, and the like. In some examples, the patient endpoint 110 can be a remotely operated robot that is controlled by the physician 118 through the physician endpoint 124.

In one embodiment, the patient endpoint 110 may include a patient-side audio receiver 112 (e.g., microphone) and a patient-side video receiver (e.g., camera) 113. The physician endpoint 124 may likewise include a physician-side audio receiver 126 and a physician-side video receiver 127. The patient-side audio/video receivers 112, 113 and the physician-side audio/video receivers 126, 127 may facilitate two-way video/audio communication between the patient 108 and the physician 118, as well as provide audio/video data to a processing server 128 via a respective endpoint 110, 124 over the communication network 106. The processing server 128 may be a remotely connected computer server 122. In some examples, the processing server 128 may include a virtual server and the like provided over a cloud-based service, as will be understood by a person having ordinary skill in the art.

The physician 118 may retrieve and review an EMR and other medical data related to the patient 108 from a networked records server 116. The records server 116 can be a computer server 120 remotely connected to the physician endpoint 124 via the communication network 106 or may be onsite with the physician 118 or the patient 108.

In addition to patient audio, video, and EMR, the physician 118 can receive diagnostic or other medical data from the patient 108 via a medical monitoring device 114 connected to the patient 108 and connected to the patient endpoint 110. For example, a heart-rate monitor may be providing cardiovascular measurements of the patient 108 to the patient endpoint 110 and on to the physician 118 via the communication network 106 and the physician endpoint 124. In some examples, multiple medical monitoring devices 114 can be connected to the patient endpoint 110 in order to provide a suite of data to the physician 118. The processing server 128 can intercept or otherwise receive data transmitted between the physician environment 104 and the patient environment 102.

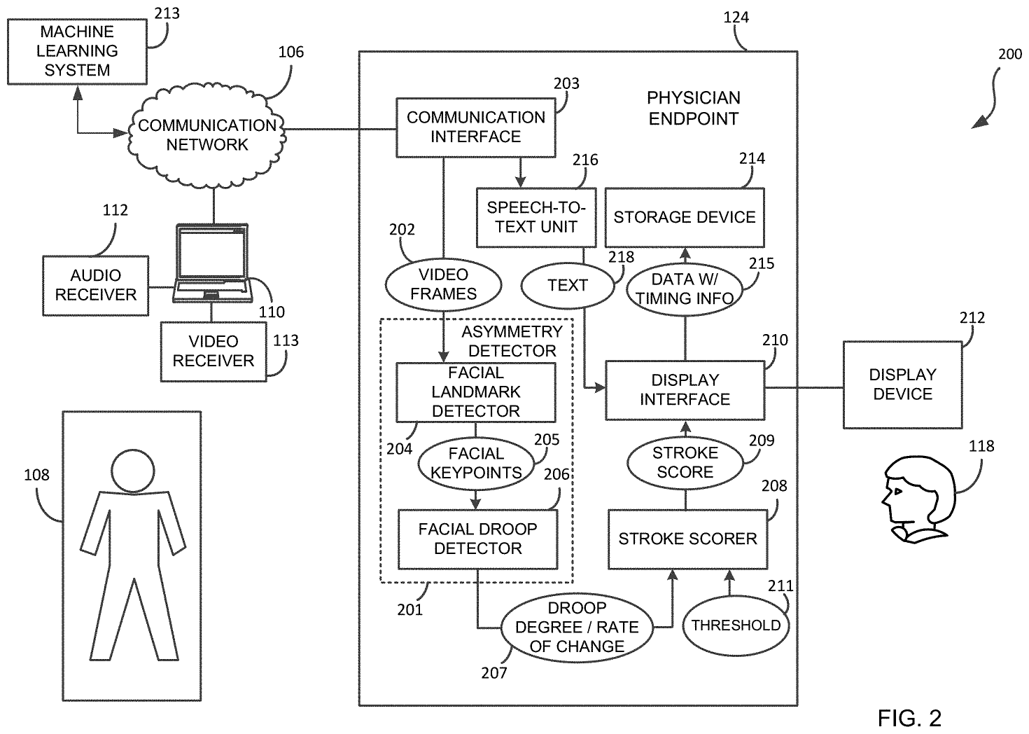

FIG. 2 is a schematic diagram of a system 200 for automated stroke scoring in a telehealth consultation. The system 200 may employ the telehealth system 100 shown in FIG. 1. In one embodiment, the video receiver 113 (e.g., camera) in proximity to the patient 108 may capture one or more video frames 202 showing the patient's face, including, in one embodiment, at least the patient's eyes and lips. The video frames 202 may include a series of 2D or 3D still images (i.e., key frames) or may include a video stream compressed using a proprietary or standard compression scheme, such as H.264, MPEG-4, MPEG-2, or the like.

The video frames 202 are sent by the patient endpoint 110 via the communication network 106 to the physician endpoint 124. While the following disclosure will often refer to the communication network 106 in the singular, the term is intended to broadly encompass one or more computer networks of the same or different type. Furthermore, while various components are depicted within the physician endpoint 124 in FIG. 2, those of skill in the art will recognize that the components could be implemented by one or more local or remote (cloud-based) servers or devices or combinations thereof. Accordingly, the illustrated components and accompanying functions should not be construed as being limited to components of (or performed by) the physician endpoint 124.

A communication interface 203 receives the video frames 202 from the communication network 106, performing any necessary network management, decryption, and/or decompression of the video frames 202. The communication interface 203, like other illustrated components of the system 200, may be implemented as one or more discrete functional components using any suitable combination of hardware, software, and/or firmware.

The communication interface 203 may provide the decrypted and/or decompressed video frames 202 to a facial landmark detector 204 that automatically identifies a set of facial keypoints 205 in at least one of the one or more video frames 202. As described more fully below, the facial keypoints 205 may include, for example, at least one point on each eye of the patient and at least one point on opposite sides of the patient's lips, although additional points may be used in various embodiments.

The facial landmark detector 204 may include (or have access to via the communication network 106) a machine learning system 213, such as a deep learning neural network. In the illustrated embodiment, the machine learning system 213 is depicted as separate from the facial landmark detector 204. However, in other embodiments, the machine learning system 213 may be a component of facial landmark detector 204. The machine learning system 213 may implemented within (or execute on) the physician endpoint 124, a remote server or device, and/or any combination thereof.

In one embodiment, the machine learning system 213 is a fully convolutional neural network based on heat map regression. The neural network may be trained, for example, on hundreds of thousands of facial data samples from a database, such as the LS3D-W database. The facial keypoints 205 may be annotated in one or both of 2D and 3D coordinates. In one embodiment, the facial landmark detector 204 is capable of detecting sixty-eight (68) or more different facial keypoints 205 on a human face. Moreover, the facial landmark detector 204 may be able to predict both the 2D and 3D facial keypoints 205 in a face. Facial landmark detectors 204 and/or machine learning systems 213 of the type illustrated are available from a number of sources, including OPENFACE, available from Carnegie Mellon University and available under the Apache 2.0 License.

The facial landmark detector 204 may provide the facial keypoints 205 to a facial droop detector 206. As described in greater detail hereafter, the facial droop detector 206 automatically calculates a degree of facial droop 207 by calculating a first line between each eye point, calculating a second line between each lip point, and calculating an angle between the first line and the second line, which angle serves as an indicator of facial asymmetry or droop 207. In one embodiment, the facial droop detector 206 determines a rate of change of the degree of facial droop 207 over the course of a consultation, such as a telehealth session between the patient 108 and the physician 118.

In one embodiment, the facial droop detector 206 determines a degree of facial droop 207 at a first time point when the patient's face is in a neutral position. Thereafter, the physician 118 may instruct the patient 108 to smile. The facial droop detector 206 may then determine a degree of facial droop 207 at a second point in time when the patient is smiling. In general, facial droop 207 is more pronounced when the patient is smiling, and the amount of change in facial droop 207 that occurs, as well as the rapidity of the change, may be diagnostic of a stroke, as well as stroke severity.

A stroke scorer 208 determines a stroke score 209 from the degree and/or rate of change of facial droop 207 and/or other inputs. In one embodiment, the stroke score 209 may include the calculated angle between the first line and the second line. In other embodiments, the stroke score 209 may be a probability, a percentage chance or other indicator of likelihood, and/or a function of the calculated angle with respect to threshold 211 and/or other inputs or parameters. For example, an angle of zero or approximately zero may indicate a high degree facial symmetry, which the stroke scorer 208 might determine a low stroke score 209 suggesting that a stroke is unlikely, whereas an angle exceeding a threshold 211 of 2.5 degrees may be given a moderate to high stroke score 209 indicating that the patient 108 likely experienced (or is undergoing) a stroke. In one embodiment, multiple thresholds 211 and/or functions may be provided, which may be determined experimentally and/or using a machine learning system.

As described in greater detail below, the degree and/or rate of change of facial droop 207 may only be one of a plurality of inputs based on the National Institutes of Health Stroke Scale (NIHSS). For example, the stroke scorer 208 may also receive as an input the patient's level of dysarthria (i.e., slurred or slow speech) or ataxia (i.e., lack of voluntary coordination of muscle movements that can include gait abnormality, and abnormalities in eye movements), each of which may be used to formulate the stroke score 209 in certain embodiments.

In one embodiment, the stroke score 209 may include an indication of stroke severity based on the rate of change of the degree of facial droop 207 as determined by the facial droop detector 206. For example, if, during the course of a consultation, the patient's facial droop 207 worsens, the stroke scorer 208 may indicate that the stroke is severe and/or assess the severity of the stroke quantitatively based on the rate of change.

Thereafter, the stroke score 209 may be provided to display interface 210 for display to the physician 118 on a display device 212, such as a computer monitor or augmented reality (AR) display. The latter may be used even when the physician 118 is in the same room as a patient 108, as it provides a quantitative assessment that could aid in a stroke diagnosis.

In one embodiment, the stroke score 209 may be simultaneously displayed with the one or more video frames 202, allowing the physician 118 to observe the patient 108 concurrently with the calculated stroke score. In addition, one or more of the facial keypoints 205, eye/lip lines, droop degree 207, rate of change of droop degree 207, threshold 211, and/or other inputs/calculations may be selectively superimposed upon the video frames 202 if desired by the physician 118 to better visualize the how the stroke score 209 was generated.

The facial landmark detector 204, the facial droop detector 206, and the stroke scorer 208 may continuously evaluate incoming video frames 202 received by the communication interface 203 in order to provide a series of real-time stroke scores 209, which may be displayed on the display device 212. Accordingly, the physician 118 can monitor the progression of a possible stroke, both visually and quantitatively.

In one embodiment, all of the data provided to the physician 118 via the display device 212 may be additionally and/or selectively stored on a storage device 214, such as a local hard disk drive or remote server, for subsequent retrieval and display. This may include, for example, one or more of the video frames 202, facial keypoints 205, eye/lip lines, degrees and/or rates of change of facial droop 207, thresholds 211, audio information (including text transcriptions) received and/or transmitted via the communication interface 203, and/or other inputs/calculations along with timing information 215 to indicate when each piece of data was received, generated, and/or calculated to permit subsequent review/playback by the physician 118 in a time-synchronized manner.

The system 200 may further include a speech-to-text unit 216, which may convert spoken audio communicated between the patient 108 and/or physician 118 via the communication interface 203 into readable text 218. The system may distinguish among participants using voice recognition techniques. The speech-to-text unit 216 may process audio via one or more neural networks or preprocessed by various services. For example, the audio may be first fed through a trained speech-to-text network such as AMAZON® TRANSCRIBE® OR NUANCE® DRAGON® and the like.

The text 218 may be displayed, in one embodiment, on the display device 212 and/or stored in the storage device 214 with timing information 215 to permit subsequent display and/or synchronization thereof with other data from a patient session stored in the storage device 214. In one embodiment, the text 218 may allow a physician 118 to note, for example, when the patient 108 was asked to smile or perform other tasks, as well as any spoken responses by the patient 108.

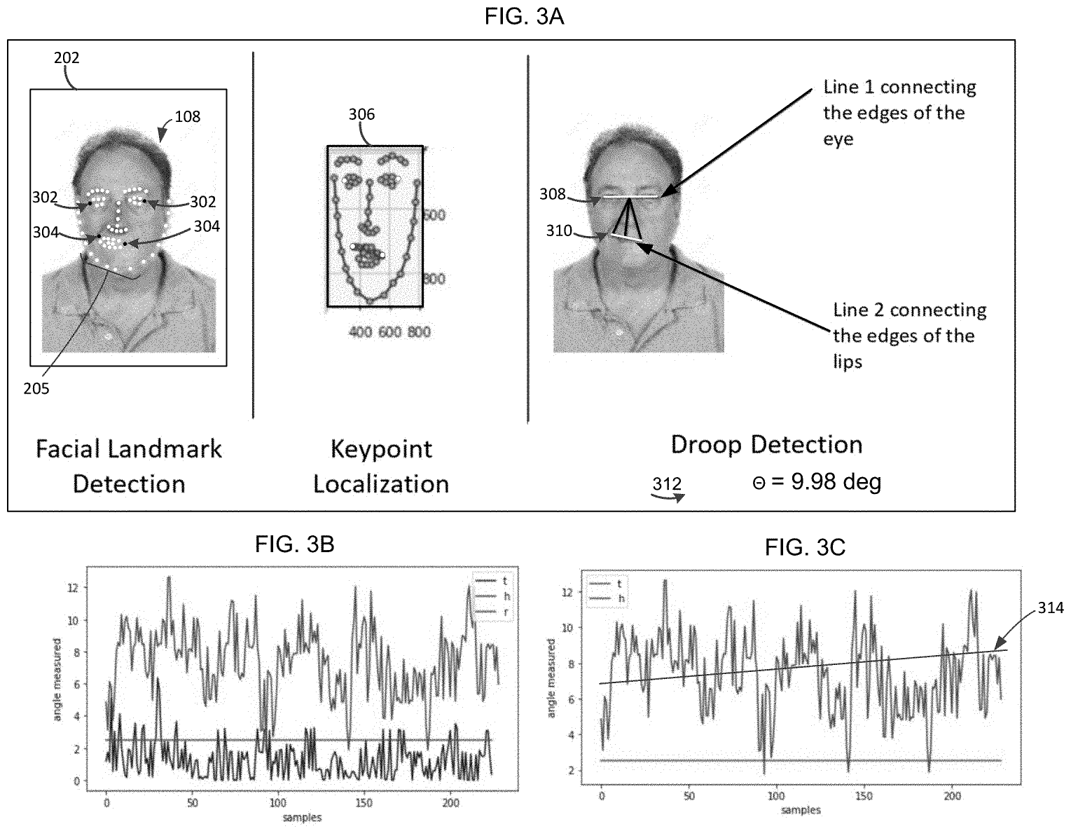

FIG. 3A illustrates a process of facial landmark detection and facial droop detection, which may be performed, for example, by the facial landmark detector 204 and facial droop detector 206 of FIG. 2. With continuing reference to FIG. 2, decompressed video frames 202 are received by facial landmark detector 204, which automatically identifies a set of facial keypoints 205 in at least one of the one or more video frames 202. As described more fully below, the facial keypoints 205 may include at least one eye point 302 on the outer edge of each eye and at least one lip point 304 on the outermost opposite sides of the patient's lips, although additional points may be used in various embodiments.

The facial landmark detector 204 may localize the facial keypoints 205 within a common coordinate system, such as the depicted 2D coordinate system 306. However, a 3D coordinate system (not shown) may be used in some embodiments.

In one embodiment, the facial droop detector 206 calculates a first line 308 (i.e., eye line) connecting the eye points 302 and a second line 310 (i.e., lip line) connecting the lip points 304. Other lines may be calculated, as shown, which can also be used to detect various forms of facial asymmetry and/or droop.

In one embodiment, the first line 308 is calculated according to the equation:

$$E = \{ ( x , y ) \vert \frac{{y}_{e 0} - {y}_{e 1}}{{x}_{e 0} - {x}_{e 1}} ( x - {x}_{e 0} ) + {y}_{e 0} = y \} \qquad Eq . 1$$

while the second line 310 is calculated according to the equation:

$$L = \{ ( x , y ) \vert \frac{{y}_{l 0} - {y}_{l 1}}{{x}_{l 0} - {x}_{l 1}} ( x - {x}_{l 0} ) + {y}_{l 1} = y \} , \qquad Eq . 2$$

where E is the line joining the eyes with e0 and e1 being the outermost points of the eyes, and L is the line joining the eyes with l0 and l1 being the outermost points of the lips. In other coordinate systems, such as 3D or polar coordinate systems, different equations would be used as understood by those of skill in the art.

In one embodiment, the facial droop detector 206 calculates an angle 312, depicted by the letter θ, between the first line 308 and the second line 310 according to the equation:

$$θ = {tan}^{- 1} \vert \frac{{m}_{e -} {m}_{l}}{1 - {m}_{e} {m}_{l}} \vert \qquad Eq . 3$$

where me and mi are slopes of the eye line 308 and lip line 310, respectively. In different coordinate systems, other equations would be used.

FIG. 3B is a graph of the measured degree of facial droop over a time sequence of sampled video frames. The y-axis indicates the angle 312 shown in FIG. 3A. The line, h, corresponds to a normal (non-stroke) pattern where the angle 312 generally lies below certain threshold 211 (shown in FIG. 2), such as 2.5 degrees, which is represented by the line, r, in the graph. By contrast, the line, t, represents an abnormal pattern likely indicative of a stroke.

FIG. 3C is another graph illustrating a temporal assessment of facial asymmetry. As previously noted, the progression of facial asymmetry during a consultation (or longer duration) is an indicator of stroke severity. In one embodiment, the line 314 shows the magnitude and rate of change in the angle 312 over a period of time represented by the sequence of video frames, which may be used to diagnose a stroke and/or the severity of the stroke.

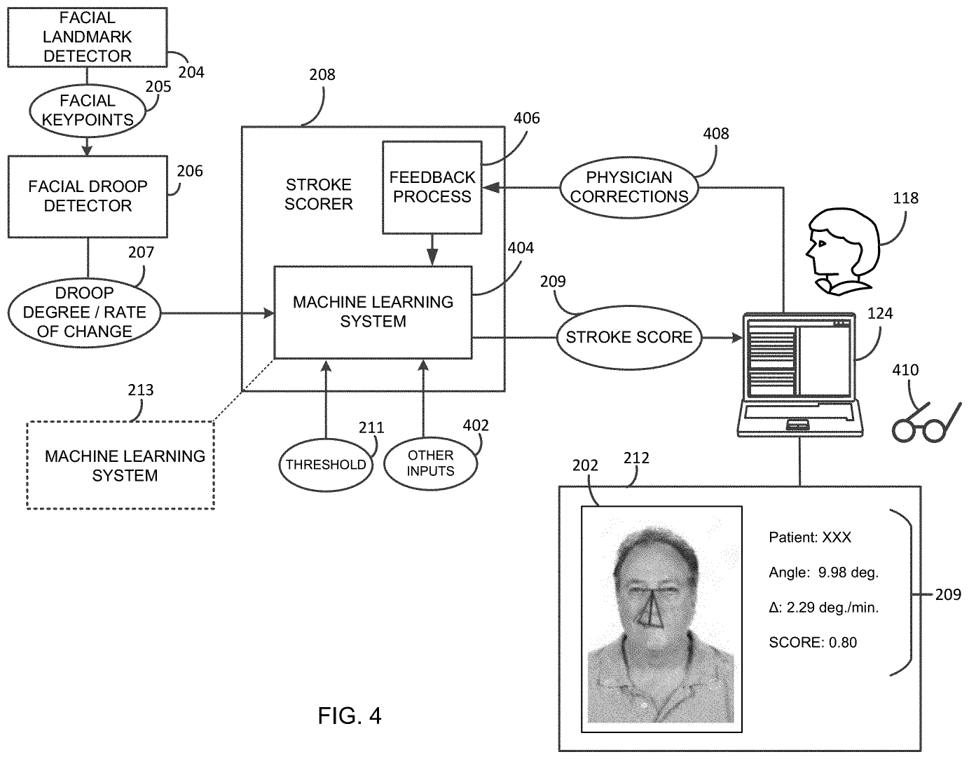

Referring to FIG. 4, and with continuing reference to FIGS. 2 and 3, the facial droop detector 206 may provide to the stroke scorer 208 the degree (and/or rate of change) of facial droop 207. The degree of facial droop 207 may comprise the angle 312 calculated by the facial droop detector 206, the rate of change of the angle 312 over time, and/or other information.

In one embodiment, the stroke scorer 208 compares the degree and/or rate of change of facial droop 207 to one or more threshold values 211. For example, if the degree of facial droop 207 is greater than 2.5 degrees, the stroke scorer 208 may output a moderate or high stroke score 209 indicating that the patient 108 has likely experienced (or is currently experiencing) a stroke. By contrast, a degree of facial droop 207 that is zero degrees or approximately zero degrees may result in a low stroke score 209 indicating that a stroke is unlikely.

As previously noted, the threshold value(s) 211 may be calculated experimentally and may be static or dynamic or rely on other variables. For example, the stroke scorer 208 may receive additional inputs 402, including demographic information and/or inputs based on the National Institutes of Health Stroke Scale (NIHSS). For example, the stroke scorer 208 may also receive indications of the patient's level of dysarthria (i.e., slurred or slow speech) or ataxia (i.e., lack of voluntary coordination of muscle movements that can include gait abnormality, speech changes, and abnormalities in eye movements), each of which could be used to formulate the stroke score 209.

Furthermore, the stroke scorer 208 may include (or have access to via the communication network 106 of FIG. 1) a machine learning system 404, such as a deep learning neural network, which may be the same as (or separate from) the machine learning system 213 shown in FIG. 2. The machine learning system 404 may combine various thresholds 211 or other inputs 402 with the degree and/or rate of change of facial droop 207 in order to determine the stroke score 209.

In one embodiment, the machine learning system 404 may be updated by a feedback process 406 in response to physician corrections 408 and/or other training data. For example, the physician 118 may note that the machine learning system 404 provided a high stroke score 209 in a case where the patient is not currently suffering a stroke. Through the feedback process 406, the machine learning system 404 may update its internal model(s) and provide different weights to various inputs, thereby improving the accuracy of the stroke score 209 in the future.

The feedback process 406 may update the machine learning system 404, using, for example, a gradient descent algorithm and back propagation and the like as will be apparent to a person having ordinary skill in the art. In some examples, the machine learning system 404 may be updated in real time or near real time. In other examples, the machine learning system 404 may perform model updates as a background process on a mirror version of the machine learning system 404 and directly update the machine learning system 404 once the mirror version has converged on an updated model. In still other examples, the feedback process 406 may perform updates on a schedule or through a batch process. The updates can be performed on a singular device or may be performed across parallelized threads and processes and the like.

As illustrated, the stroke score 209, which may include or supplement the angle and/or rate of change of facial droop when displayed on the display device 212 along with the video frame(s) 202 and/or other data. As previously described, the facial keypoints, eye lines, lip lines and/or other information may be superimposed upon the video frame(s) 202 in order to provide the physician 118 with a graphical view of how the stroke score 209 is being determined. In some cases, this may allow the physician 118 to correct, via the feedback process 406, detection errors by the facial landmark detector 204 and/or stroke scorer 208, which may occur, for example, in the case of patient racial types for which the machine learning system 213 and/or 404 have been inadequately trained.

In one embodiment, the video frames 202 and stroke score 209 may be shown on an augmented reality (AR) or virtual reality (VR) headset 410. AR and VR headsets 410 are available from a number of manufacturers, including OCULUS VR of Menlo Park, Calif., and MAGIC LEAP of Sunnyvale, Calif..

In the case of an AR headset 410, the physician 118 may be able to examine the patient in person while still obtaining real-time stroke scores 209 calculated by the machine learning systems 213 and/or 404. This may increase the accuracy of the physician's diagnosis, particularly if the facial droop detector 206 is able to identify subtle changes in the degree and/or rate of change of facial droop 207 that would otherwise be difficult to detect by the physician 118 while focused on other aspects of patient care.

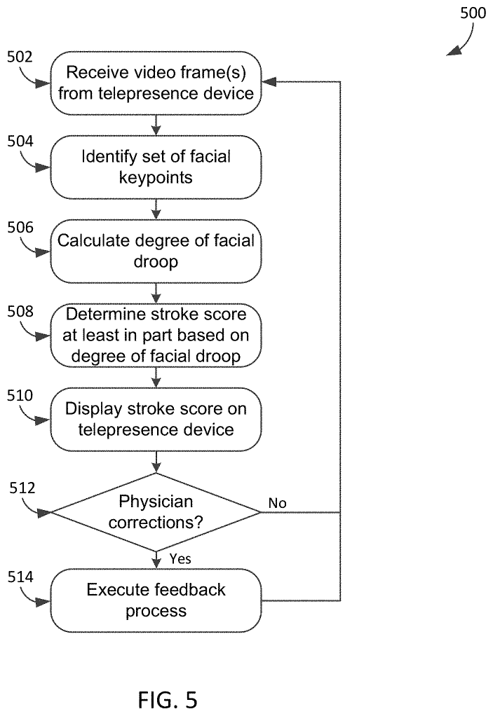

FIG. 5 is a flowchart of a method 500 for automated stroke scoring based on a measurement of facial asymmetry. Initially, one or more video frames are received 502 from a telepresence device in a patient environment. The telepresence device could be a robotic endpoint, although the method 500 is not limited in that respect. The video frames may show a patient's face including, for example, the patient's eyes and lips.

Thereafter, a set of facial keypoints is automatically identified 504 within the one or more video frames. The set of facial keypoints may include, for example, at least a point on each eye of the patient and at least one point on opposite sides of the patient's lips. The facial keypoints may be automatically identified by a machine learning system, such as, for example, a deep learning neural network.

In one embodiment, the degree and/or rate of change of facial droop is then automatically calculated 506, for example, by calculating a first line between each eye point, calculating a second line between each lip point, and calculating an angle between the first line and the second line.

Based at least in part on the degree and/or rate of change of facial droop, a stroke score is automatically determined 508. The stroke score may be determined, in one embodiment, using a machine learning system, such as a deep learning neural network. Alternatively, or in addition, the stroke score may be calculated based on the calculated angle with reference to one or more threshold values and/or other inputs.

Thereafter, an indication of the stroke score is displayed 510 to a physician. The stroke score may be displayed, for example, with the one or more of the input video frames and/or other data on a telepresence device of the physician.

A determination 512 is then made whether any physician corrections have been provided. If so, a feedback process 514 is executed, by which one or more machine learning systems are updated or refined to incorporate the physician corrections. In either case, the method 500 returns to receive 502 and process the next video frame(s).

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the methods can be rearranged while remaining within the disclosed subject matter. The accompanying method claims present elements of the various steps in a sample order, and are not necessarily meant to be limited to the specific order or hierarchy presented.

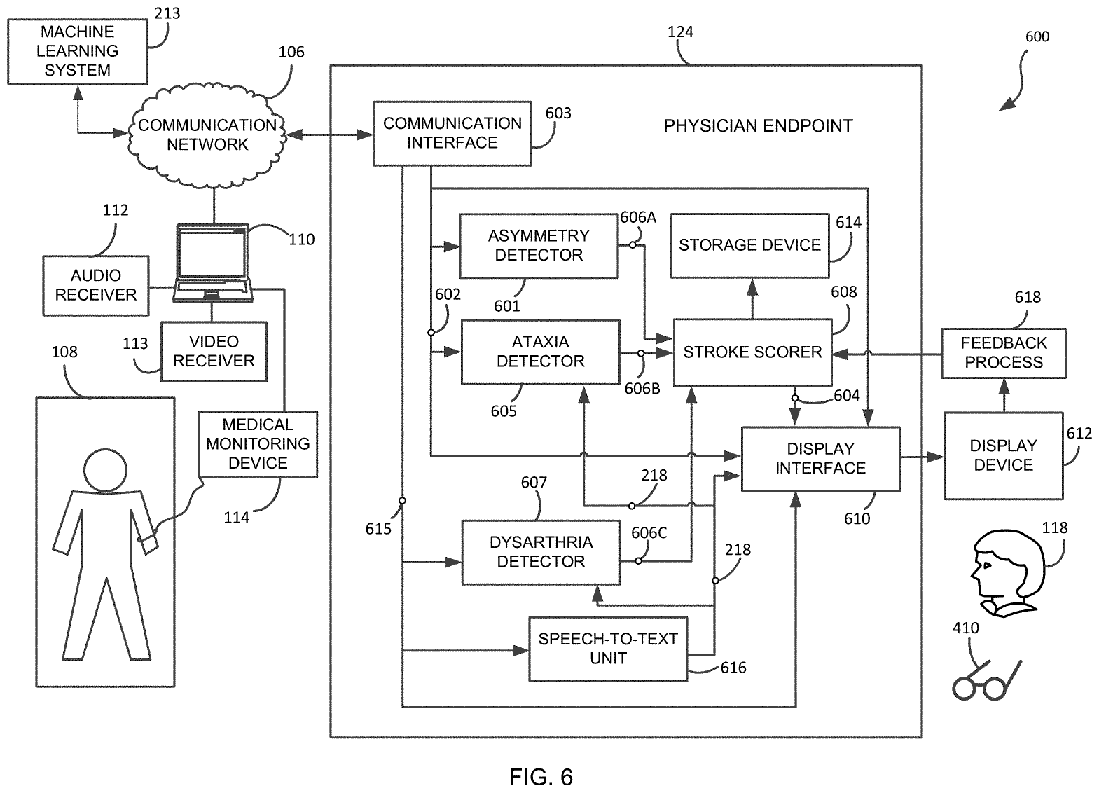

FIG. 6 is a schematic diagram of another embodiment of a system 600 for automated stroke scoring. The system 600 may include an asymmetry detector 601, similar to the asymmetry detector 201 of FIG. 2, which receives a video stream 602 including a sequence of video frames from a video receiver 113 (e.g., camera) in proximity to the patient 108.

The video stream 602 may be received by the asymmetry detector 601 via a communication interface 603, which may be similar to the communication interface 203 of FIG. 2. In addition, the system 600 may include a stroke scorer 608, a display interface 610, a display device 612, a storage device 614, and a speech-to-text unit 616, each of which may be similar to related components (208, 210, 212, 214, and 216) of FIG. 2 with such differences as discussed below.

As previously discussed, the stroke scorer 608 may rely on various inputs in calculating an overall stroke score 604, which may be displayed to the physician 118 using the display interface 610 and display device 612 (and/or AR/VR headset 410). The asymmetry detector 601 may automatically provide the stroke scorer 608 with a first stroke likelihood 606A based on measurement of the patient's facial droop as discussed with reference to FIGS. 2-5. In one embodiment, once a telehealth consultation has been established, the asymmetry detector 601 may automatically and continuously evaluate the patient 108 for signs of facial droop based on the video stream 602 and provide the first stroke likelihood 606A to the stroke scorer 608.

In one embodiment, the system 600 further includes an ataxia detector 605 that automatically provides the stroke scorer 608 with a second stroke likelihood 606B based on a measurement of the patient's limb weakness. As described in greater detail below, the measurement of limb weakness may be a function of the movement velocity of a particular limb of the patient 108 over a time interval during which the patient 108 is instructed to keep the limb motionless. Separate velocity measurements for individual limbs may be provided and/or a summation (or other function) of the limb velocities of multiple limbs. As with the asymmetry detector 601, once a telehealth consultation has been established, the ataxia detector 605 may automatically and continuously evaluate the patient 108 for signs of limb weakness based on the video stream 602 and provide the second stroke likelihood 606B to the stroke scorer 608.

The system 600 may further include a dysarthria detector 607 that automatically provides the stroke scorer 608 with a third stroke likelihood 606C based on a measurement of the patient's slurred speech. The dysarthria detector 607 and speech-to-text unit 616 may accept as input an audio stream 615 provided by the audio receiver 112 (e.g., microphone) in proximity to the patient 108. As with the other detectors 601, 605, the dysarthria detector 607 may automatically and continuously evaluate the patient 108 for signs of slurred speech based, in this case, on the audio stream 615 and provide the third stroke likelihood 606C to the stroke scorer 608.

The detectors 601, 605, and 607 may receive various other inputs, including, without limitation, vital sign information from the medical monitoring device 114, text from the speech-to-text unit 616, one or more thresholds, selections and/or other inputs provided by the physician 118, the output of one or more machine learning systems 213, and the like. For example, in some embodiments, the ataxia detector 605 and dysarthria detector 607 may each receive transcribed text 218 from the speech-to-text unit 616.

In some embodiments, the stroke scorer 608 may receive input from additional detectors or other sources. For example, a pupillometry unit (not shown), may evaluate the video stream 602 to identify signs of a posterior fossa stroke using eye tracking to determine the patient's ability or inability to move their eyes as directed by the physician 118. Likewise, the stroke scorer 608 may receive an estimate of the patient's aphasia, i.e., a loss of the ability to understand or express speech, as well as vital sign information (e.g., blood pressure), provided by medical monitoring device 114.

The various stroke likelihoods 606A, 606B, 606C provided by the detectors 601, 605, 607, respectively, may be represented as confidence levels, odds, percentages, and/or other calculations (e.g., droop degree). Furthermore, the various likelihoods need not all be expressed using the same metrics or units, although, in certain embodiments, each of the likelihoods may be represented with a confidence level expressed as a percentage between 0 and 100.

In calculating an overall stroke score 604 to provide to the display interface 610, the individual likelihoods of stroke may be variously weighted by stroke scorer 608. For example, in a system 600 including six inputs (left arm motion, right arm motion, left leg motion, right leg motion, slurred speech, and facial asymmetry), a weighted stroke score (S) may be calculated according to the equation,

S=(w_1*l_arm)+(w_2*r_arm)+(w_3*r_leg)+(w_4*r_leg)+(w_5*slurred_speech)+(w_n*facial_asym) Eq. 4

where w_1 . . . w_n are a set of expert-defined weights for assessing the combined effect of each input, which may be determined experimentally and/or with the assistance of the machine learning system 213.

In the latter case, the stroke scorer 608 may include or have access to the machine learning system 213, as described with reference to FIG. 2, which may be embodied, for example, as a deep learning neural network. For an embodiment including a neural network, a feedback process 618 may be provided for updating the internal models of neural network based on physician corrections and/or other training data, as described with reference to FIG. 4.

Table 1 illustrates an empirical evaluation of a stroke scorer, similar to the stroke scorer 608 of FIG. 6, for different weights for each input. The shorthand notations for weights of each input are WFA-Facial Asymmetry, WSS-Slurred Speech, WRA-Right Arm, WLA-Left Arm, WRL-Right Leg, and WLL-Left Leg.

TABLE 1

| WFF | WSS | WRA | WLA | WRL | WLL | Accuracy |
|---|---|---|---|---|---|---|
| 0.166 | 0.166 | 0.166 | 0.166 | 0.166 | 0.166 | 57.14% |
| 0.5 | 0.1 | 0.1 | 0.1 | 0.1 | 0.1 | 42.85% |
| 0.1 | 0.5 | 0.1 | 0.1 | 0.1 | 0.1 | 42.85% |
| 0.33 | 0.33 | 0.082 | 0.082 | 0.82 | 0.82 | 71.41% |
| 0.35 | 0.33 | 0.08 | 0.08 | 0.08 | 0.08 | 85.714% |

All of the data received and/or produced by the stroke scorer 608, as well as the video stream 602 (and/or individual video frames 202), the audio stream 615, the text output from the speech-to-text unit 616, and/or any calculations may be stored in the storage device 614 with relevant timing information to permit subsequent retrieval and review. Likewise, all of the foregoing may be displayed to the physician in real-time via the display interface 610 on a display device 612, such as a computer monitor, and/or a virtual or augmented reality headset 410.

In FIG. 6, various components are illustrated as being integral to physician endpoint 124, which may be embodied as a desktop computer, laptop, or the like. However, any of the illustrated components could be implemented in one or more remote servers and/or separate devices that are in communication with the physician endpoint 124.

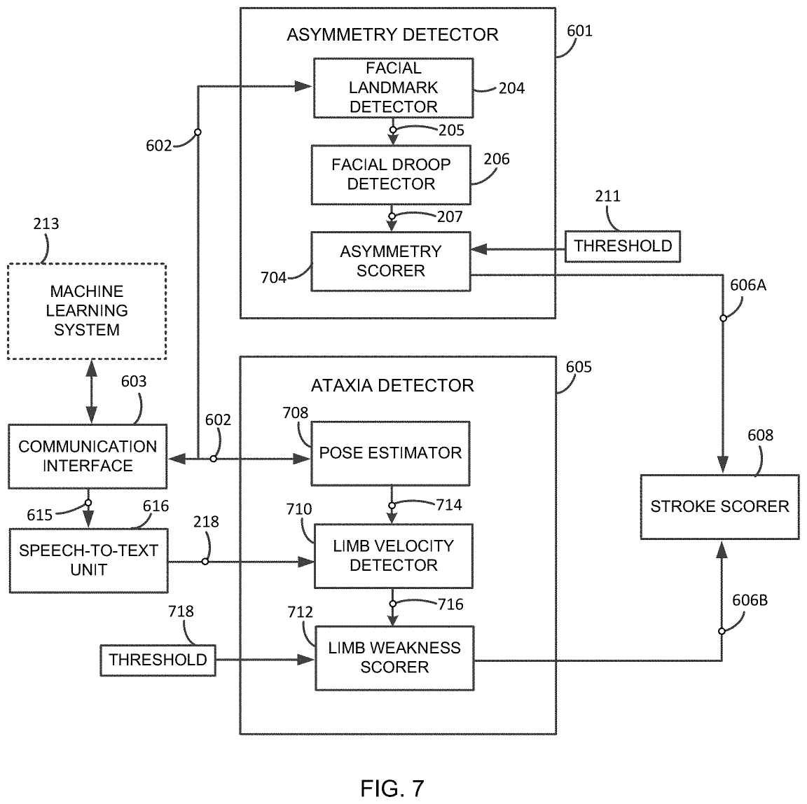

FIG. 7 provides additional details of the asymmetry detector 601 and ataxia detector 605. As previously noted, the asymmetry detector 601 may operate similarly to the asymmetry detector 201 of FIG. 2. For example, the asymmetry detector 601 may receive as input a video stream 602 including a sequence of video frames provided by the communication interface 603.

However, in this embodiment, the asymmetry detector 601 provides the stroke scorer 608 with a first stroke likelihood 606A based on a measurement of facial asymmetry, unlike the asymmetry detector 201 of FIG. 2, which is illustrated as providing the degree (and/or rate of change) of facial droop 207 to the stroke scorer 208. Of course, in other embodiments, either or both inputs may be provided to the stroke scorer 608 in addition to other information.

The asymmetry detector 601 may include a facial landmark detector 204, which provides a set of facial keypoints 205 to a facial droop detector 206, which, in turn, provides the degree (and/or rate of change) of facial droop 207 to an asymmetry scorer 704. In this embodiment, the asymmetry scorer 704 may operate in much the same way as the stroke scorer 208 of FIG. 2 in determining the first stroke likelihood 606B based on a single stroke factor, i.e., facial asymmetry. This may be accomplished with reference to one or more thresholds 211, as previously described. Likewise, the asymmetry scorer 704 may include or have access to a machine learning system 213 and otherwise operate in a manner similar to the stroke scorer 208 of FIG. 2.

The ataxia detector 605 may likewise receive as input a video stream 602 provided by the communication interface 603. In turn, the ataxia detector 605 provides the stroke scorer 608 with a second stroke likelihood 606B of stroke based on a measurement of limb weakness (ataxia).

In one embodiment, limb weakness is detected by continuously monitoring the movement of the patient's limbs, i.e., arms and legs. Limb velocity is used, in one embodiment, as a measure of limb weakness. During a stroke consultation, a physician asks the patient to raise one or more limbs and hold them motionless for a given amount of time. A non-stroke patient should be able to maintain the outstretched position of their limbs for a period of time with little or no visible motion.

In one embodiment, the ataxia detector 605 includes a pose estimator 708, a limb velocity detector 710, and a limb weakness scorer 712. Initially, the pose estimator 708 automatically identifies body keypoints 714 in at least one frame of the video stream 602. The body keypoints 714 may include points on one or more of the patient's limbs and, in particular, at the joints of those limbs.

The body keypoints 714 may be identified using a machine learning system 213, such as a neural network, in the same manner that the facial keypoints 205 were determined in FIG. 2. The machine learning system 213 may be a component of the ataxia detector 605 or accessed remotely via the communication interface 603, as shown.

In one embodiment, the pose estimator 708 may employ OpenPose, available from Carnegie Mellon University, to detect both pose and limb movement. OpenPose uses a non-parametric approach to estimate body parts for individuals in a given image, and is relatively robust to occlusion of one or two limbs. The algorithm is also robust to different environments and can also predict the poses of multiple individuals in a frame. In one version, the entire body is divided into 25 different joints, although more or fewer joints (defined by body keypoints 714) may be identified in different embodiments.

Using the body keypoints 714, the limb velocity detector 710 determines a movement velocity 716 of a limb over a time interval in which the patient is instructed to keep the limb motionless. The movement velocity 716 of the patient's limb may be continuously calculated during the stroke consultation and may be expressed, in one embodiment, as the sum of the velocities for all of the joints in the limb.

The limb velocity detector 710 may determine the movement of each joint per unit of time (e.g., second) with the assumption that the overall velocity of the limbs remains close to 0 when the patient is asked to hold their limb straight for a period of time, e.g., 5 seconds. If there is a high change in the velocity of the limb during the period, it implies that the patient may have weakness in the given limb.

The cumulative velocity, φⱼ, may be calculated using the equation:

φⱼ=Σₗ₌₁^{l=n} posKₗ(t)−posKₗ(t−1) Eq. 5

which denotes the cumulative velocity of the jth limb which is a sum of the displacement of all I joints of the limb over unit time. The term poski(t) denotes the position of the Ith joint over time t.

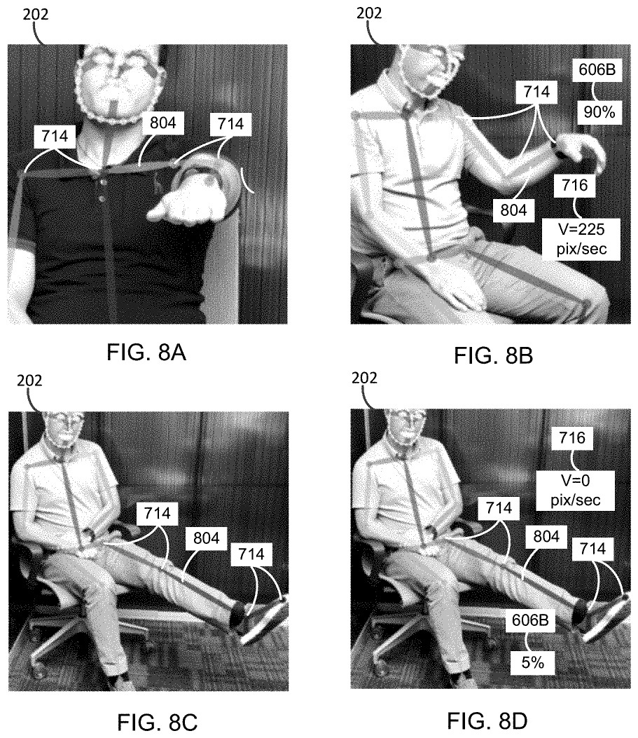

As an example, referring also to FIG. 8A and 8B, the movement velocities 716 of the arm joints may be determined when the patient is asked to raise their arm. A normal subject with no limb weakness will be able to hold the limb straight for a time interval. Hence, the movement velocity 716 will be close to zero. However, as shown, a patient with arm weakness will not be able to hold their arm for the requisite time and their limb will fall or drift away from the held position before the end of the time interval. Therefore, such a subject will have a high sum of limb movement velocities 716.

In one embodiment, the time interval for measuring the limb movement velocity 716 is defined by physician input. For example, the physician may activate a particular control (not shown) at the physician endpoint to mark a point in time at which the patient raises their arm in response to a verbal instruction from the physician. The time interval may be for a set period, e.g., 5 seconds, or for a dynamic period specified by the physician.

Alternatively, the time interval for measuring limb movement velocity 716 may be automatically determined at least in part based on transcribed text 218 of the audio stream 615 between the patient and the physician, as well as limb motion detected in the video stream 602. For example, the speech-to-text unit 616 may distinguish between words spoken by the physician and the patient. When the physician instructs the patient, “raise your arm,” the resulting text 218 may be noted by the ataxia detector 605. Thereafter, limb velocity detector 710 may determine the point in time at which the patient has actually raised their arm as the beginning of the time interval. The time interval may be for a set period, e.g., 5 seconds, or for a dynamic period ending, for example, when the patient drops the limb.

Thereafter, the limb weakness scorer 712 calculates the second stroke likelihood 606B, which represents a measurement of limb weakness, as a function of the movement velocity 716 of the limb over the time interval. In one embodiment, a threshold may be provided and/or learned by the machine learning system 213 for whether, and/or to what degree, the movement velocities 716 for one or more limb(s) is consistent with a stroke.

In one embodiment, a measurement of limb weakness (i.e., second stroke likelihood 606B) may be determined for each of a plurality of limbs, e.g., left arm, right arm, left leg, and right leg. The second stroke likelihood 606B may be provided to the stroke scorer 608 for each of the patient's limbs and/or a function of multiple limbs during the consultation and/or an interval thereof.

Table 2 illustrates a performance evaluation for the output of the limb weakness scorer 712 based on different thresholds (in pixels per second).

TABLE 2

| Velocity | Left Leg | Right Leg | Left Arm | Right Arm |
|---|---|---|---|---|
| 0 pix/sec | 42.85% | 42.85% | 42.85% | 42.85% |
| 100 pix/sec | 42.85% | 71.42% | 71.42% | 71.42% |
| 150 pix/sec | 71.42% | 71.42% | 85.71% | 85.71% |
| 250 pix/sec | 71.42% | 71.42% | 100% | 100% |
| 300 pix/sec | 100% | 100% | 100% | 100% |

In one embodiment, the limb weakness scorer 712 indicates the second stroke likelihood 606B as a probably, percentage chance, confidence level and/or other indication, which may be determined experimentally and/or discovered by the machine learning system 213.

FIGS. 8A and 8B illustrate body keypoints 714 at various joints determined by the pose estimator 708, which are superimposed upon frames 202 of the video stream input. In FIG. 8A, the patient has been instructed to maintain his arm without motion in an outstretched position for a period of time. However, as shown in FIG. 8B, the patient's ataxia causes the arm to quickly droop. The limb velocity detector 710 uses the relative motion of keypoints 714 over the time interval to determine the movement velocity of the limb. Thereafter, the limb weakness scorer 712 calculates the second stroke likelihood 606B as measurement of limb weakness (ataxia). In the case of FIGS. 8A and 8B, the limb weakness scorer reports a high likelihood of stroke based on the calculated velocities. By contrast, FIGS. 8C and 8D illustrate a negative case in which the patient is able to keep his leg motionless for a prescribed time period.

As described in greater detail hereafter, the body keypoints 714 at various joints of the patient (and, optionally, one or more joint connection lines 804 connecting the body keypoints 714) may be displayed with and/or superimposed over the video frames 202 on the physician's display device, as well an indication of the calculated movement velocity 716 and/or stroke likelihood 606B for one or more limbs.

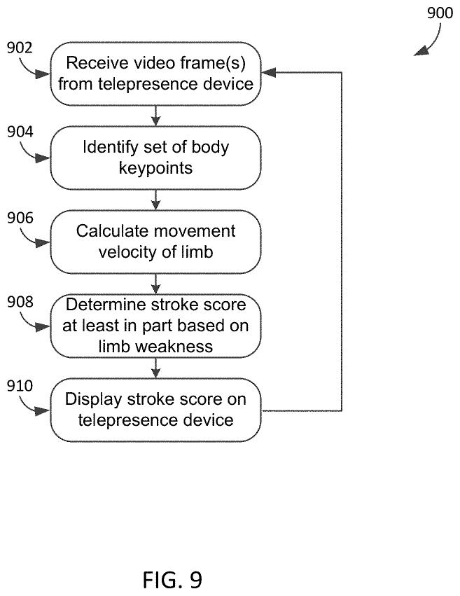

FIG. 9 is a flowchart of a method 900 for automated stroke scoring based on a measurement of ataxia. Initially, one or more video frames are received 902 from a telepresence device in a patient environment. The telepresence device could be a robotic endpoint, although the method 900 is not limited in that respect. The video frames may depict one or more of the patient limbs at time points before, during, and after the physician instructs the patient to keep the limb in an outstretched position without motion.

A set of body keypoints is automatically identified 904 within the one or more video frames. The set of body keypoints may include, for example, points at various joints of the limb(s) in question. The body keypoints may be automatically identified by a machine learning system, such as, for example, a deep learning neural network.

In one embodiment, the movement velocity of the limb(s), which is used as a measurement of limb weakness, is automatically calculated 906, for example, by calculating the sum of the velocities for all the joints in a limb at a time that the patient is instructed to keep the limb motionless.

Based at least in part on the measurement of limb weakness, a stroke score is automatically determined 908. The stroke score may be determined, in one embodiment, using a machine learning system, such as a deep learning neural network.

Thereafter, an indication of the stroke score is displayed 910 to a physician. The stroke score may be displayed, for example, with the one or more of the input video frames and/or other data on a telepresence device of the physician, such as a laptop or mobile device.

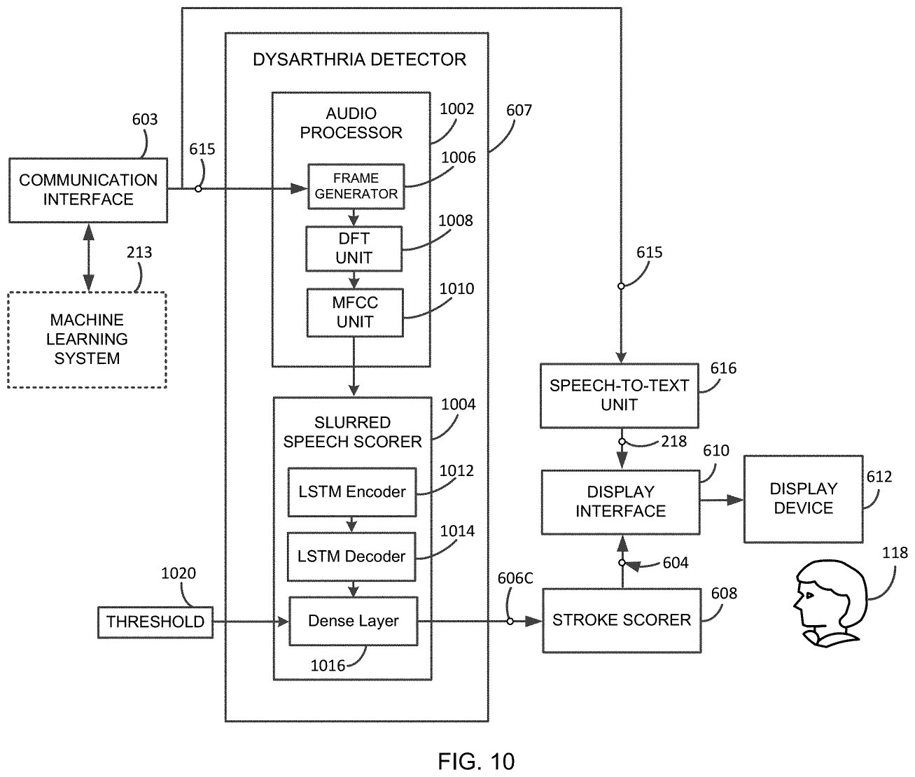

FIG. 10 provides additional details of the dysarthria detector 607 shown in FIG. 6. As previously noted, the dysarthria detector 607 may receive as input an audio stream 615 provided by the communication interface 603. In turn, the dysarthria detector 607 provides the stroke scorer 608 with third stroke likelihood 606C based on a measurement of slurred speech.

In one embodiment, the dysarthria detector 607 includes an audio processor 1002 and a slurred speech scorer 1004. The audio processor 1002 may include a frame generator 1006 that converts the audio stream 615 into speech frames of 25 ms each, although other frame sizes may be used in different embodiments. Thereafter, a DFT (Discrete Fourier Transform) unit 1008 calculates the DFT of these frames. A MFCC (Mel-Frequency Cepstral Coefficients) unit 1010 applies Mel Filter banks, which are a set of filters widely used for speech recognition tasks, followed by calculating a power spectra of each filter bank. The power spectra of each filter bank provides information about the amount of energy associated with each of the filters.

The MFCC unit 1010 then converts these filter bank energies into a log scale due to the broad range of values, after which a Discrete Cosine Transform (DCT) is applied to the log of all these energies. In one embodiment, only the top 13 coefficients in each Mel Frequency filter bank are retained excluding the δ, δδ, energy, the 0^{th} coefficient, etc. The top 13 coefficients are chosen in one embodiment because these signals carry the maximum information about the speech signal.

The resulting MFCC coefficients are then fed as an input to the slurred speech scorer 1004 to detect slurred speech. The slurred speech scorer 1004 may be embodied as a deep learning neural network that may be included as a component of the dysarthria detector 607 or may be remotely via the communication interface 603, such as the machine learning system 213.

The deep neural network of the slurred speech scorer 1004 may use an encoder and decoder structure including a LSTM (Long Short-Term Memory) encoder 1012 and a LSTM decoder 1014. LSTM is an artificial recurrent neural network architecture used in the field of deep learning. The encoder with an LSTM of unit size 100 and is used to encode the MFCC coefficients. These encoded embeddings are then fed into the LSTM decoder 1014 that consists of another LSTM of size 100 followed by a dense layer 1016 of size 50 and a softmax layer (not shown) to discriminate the given speech as slurred or non-slurred. Although the LSTM architecture is used in the illustrated embodiment, other neural network architectures could be used.

In one embodiment, the slurred speech scorer 1004 calculates the third stroke likelihood 606C, represented as a measurement of slurred speech, by comparing a first set of audio coefficients produced, for example, while the patient reads or repeats a pre-defined text, with a second set of audio coefficients previously generated using a reference sample for the pre-defined text spoken by an unimpaired individual. Thereafter, a measurement of slurred speech may be calculated as a function of the first and second sets of audio coefficients and one or more threshold values 1020.

The dysarthria detector 607 may then provide third stroke likelihood 606C to the stroke scorer 608 for calculating an overall stroke score. The stroke score may be displayed to the physician 118 using the display interface 610 and associated display device 612. In one embodiment, as described in greater detail below, the output of the dysarthria detector 607 may also be displayed along with text 218 generated by the speech-to-text unit 616 in order to assist the physician 118 in assessing the patient's dysarthria.

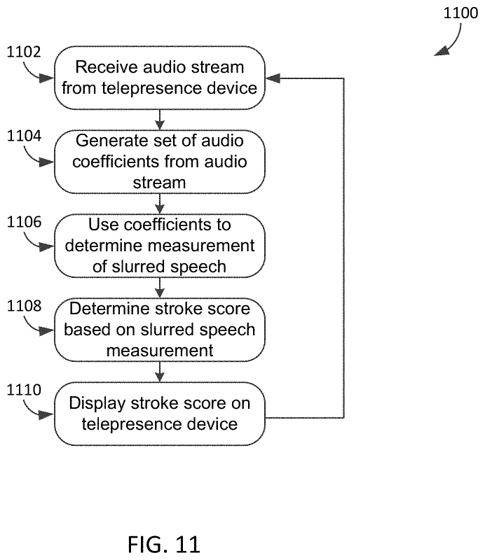

FIG. 11 is a flowchart of a method 1100 for automated stroke scoring based on a measurement of slurred speech (dysarthria). Initially, an audio stream including the patient's voice is received 1102 from a telepresence device in a patient environment. The telepresence device could be a robotic endpoint, although the method 900 is not limited in that respect.

A set of audio coefficients is then automatically determined 1104 from the audio stream. The coefficients may be automatically determined using various signal processing and speech recognition techniques, such as the application of Mel Filter banks to obtain Mel-Frequency Cepstral Coefficients (MFCCs).

In one embodiment, the coefficients are used 1106 to determine a measurement of slurred speech, after which a stroke score may be determined 1108 based on the slurred speech measurement. The measurement of slurred speech and/or stroke score may be determined, in one embodiment, using a machine learning system, such as a deep learning neural network. The indication of the stroke score is then displayed 1110 to a physician.

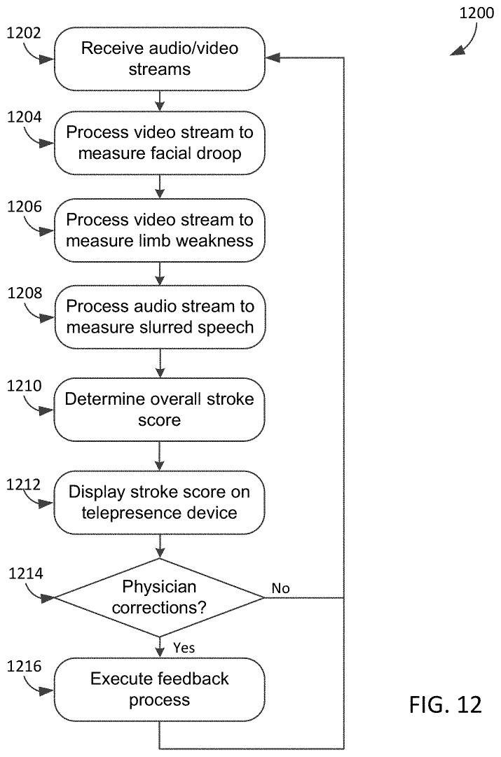

FIG. 12 is a flowchart of a method 1200 for automated stroke scoring based on a plurality of inputs generated by the detectors 601, 605, 607 of FIG. 6. Initially, audio and video streams are received 1202 from a telepresence device in a patient environment. The telepresence device could be a robotic endpoint, although the method 1200 is not limited in that respect. The video stream may include video frames that show a patient's face including the patient's eyes and lips, as well as one or more of the patient's limbs. The audio stream may include the patient's spoken voice.

In one embodiment, the video stream is processed 1204 to automatically determine a first stroke likelihood based on a measurement of facial droop. Concurrently or contemporaneously, the video stream may also be processed to automatically determine 1206 a second stroke likelihood based on a measurement of limb weakness, while the audio stream may be processed to automatically determine 1208 a third stroke likelihood based on a measurement of slurred speech. Each of the measurements of facial droop, limb weakness, and slurred speech may be determined using one or more machine learning systems, such as deep learning neural networks.

Based at least in part on the first, second, and third stroke likelihoods, a stroke scorer automatically determines 1210 an overall stroke score. The stroke score may be determined, in one embodiment, using a machine learning system, such as a deep learning neural network, which applies various weights to the first, second, and third stroke likelihoods in calculating an overall score.

Thereafter, an indication of the stroke score is displayed 1212 to a physician. The stroke score may be displayed, for example, with the video stream, audio stream, text generated from the audio stream, and/or other information, as described more fully below.

A determination 1214 is then made whether any physician corrections have been provided. If so, a feedback process 1216 is executed, by which one or more machine learning systems are updated or refined to incorporate the physician corrections. In either case, the method 1200 returns to continue receiving 1202 receiving the audio and video streams.

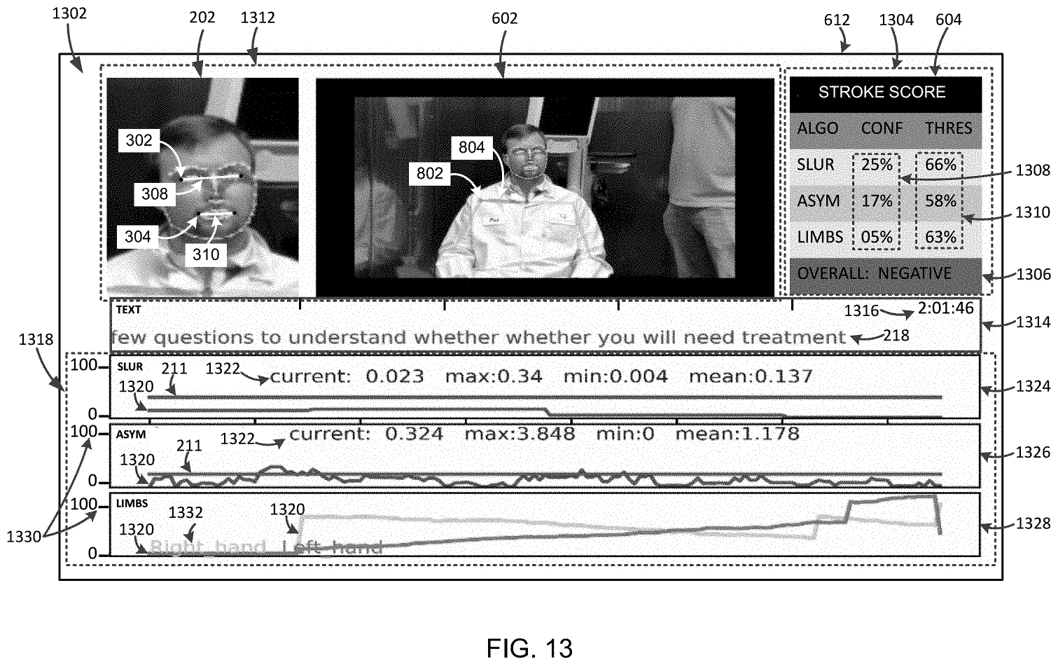

FIG. 13 shows one embodiment of an exemplary user interface 1302 displayed on a display device 612, such as a computer monitor or augmented reality display, at a physician endpoint during a stroke consultation.

In one embodiment, the user interface 1302 includes a scoring area 1304, which may be used to display a stroke score 604. The stroke score 604 may include a variety of information, including an overall stroke assessment 1306, which may be a binary (positive/negative) assessment based on one or more thresholds, as shown, or a numerical assessment, such as a percentage chance, confidence level, or the like.

The stroke score 604 may also include individual stroke likelihoods 1308 (e.g., the first, second, and third stroke likelihoods 606A-C) produced by the various detection modules in FIG. 6, such as the asymmetry detector 601, the ataxia detector 605, and the dysarthria detector 607. The individual stroke likelihoods 1308 may be expressed as a percentage chance, confidence level, or the like, together with an indication of the associated measurement (e.g., SLUR, ASYM, LIMBS).

In one embodiment, one or more thresholds 1310 may be displayed (such as the previously discussed thresholds 211, 718, 1020) that correspond to whether the respective individual stroke likelihoods 1308 are or are not indicative of a stroke. The thresholds 1310 may be the same as the threshold 211, 718, 1020 discussed above or a different set of thresholds specifically for generating the overall stroke assessment 1306. The thresholds 1310 may be established experimentally, by machine learning, and/or by the physician or another expert.

The user interface 1302 may further include a video display area 1312, which may be used to display the video stream 602 and/or individual video frames 202. In one embodiment, two separate sections of the video display area 1312 are provided—one that is focused on the patient's face and the other depicting at least a portion of the patient's body. However, both sections may be derived from the same video frame 202 and/or video stream 602. Furthermore, in another embodiment, only a single section showing the complete video frame 202 and/or video stream 602 may be provided.

As illustrated, the video frame 202 and/or video stream 602 may be superimposed with facial keypoints, such as eye points 302 and lip points 304, as well as body keypoints 714. In addition, various lines may be superimposed upon the video, such as eye lines 308, lip lines 304 and/or joint connection lines 804. The superimposed points and/or lines may be selective displayed or removed as desired by the physician.

The user interface 1302 may further include a text area 1314, which may be used to display text 218 transcribed by the speech-to-text unit 616 of FIG. 6. The text area 1314 may display the last few seconds of transcribed text 218. In one embodiment, however, if the physician clicks on or otherwise selects the text area 1314, the text area 1314 maybe be expanded in size to reveal text 218 over a longer time period and/or all of the text 218 generated since the beginning of the consultation, which the physician may scroll through, copy, mark, annotate, and/or highlight, as desired. The text area 1314 (or other areas of the user interface 1302) may further include an indication of the current time 1316 and/or the amount of time that has elapsed since the consultation began. The text 218 may scroll or otherwise be periodically replaced such that the displayed text 218 corresponds to the most recent text and/or a particular time interval represented by the text area 1314.

In one embodiment, the user interface 1302 also includes a trend area 1318, which may be used to display trend lines 1320 for each of the various stroke likelihoods generated by the detectors of FIG. 6. In one embodiment, the trend lines 1320 for each likelihood indication (e.g., slurred speech, facial asymmetry, limb ataxia) are aligned on a common time axis, which may also correspond to the time interval represented in the text area 1314. In one embodiment, one or more thresholds 1310 may be displayed as a separate line next to the associated trend lines 1320, allowing the physician to visually determine the time(s) at which each stroke likelihood exceeds or drops below the respective threshold 1310.

The trend area 1318 may also include one or more numerical indications 1322 of the stroke likelihood indication in question, including, without limitation, the current value, the maximum value over a period of time (e.g., over the consultation), the minimum value over the period of time, and/or the average (mean) value over the period of time.

The trend area 1318 may be divided into separate sections according to each stroke likelihood calculation. For example, the trend area 1318 may include a slurred speech section 1324, a facial asymmetry section 1326, and a limb ataxia section 1328, each of which may include their own trend lines 1320, thresholds 1310, and numerical indications 1322. The sections 1324, 1326, 1328 may each have a common time scale, although different time scales could be provided in some embodiments.

Furthermore, the sections 1324, 1326, 1328 may have the same or different X-axis scales 1330. In the illustrated embodiment, each scale 1330 is identical, running between zero and 100 percent, which may be the case if the detectors of FIG. 6 each produce a likelihood of stroke expressed as a percentage. However, in other embodiments, the scales 1330 may differ from section to section.

In some embodiments, as shown in the limb ataxia section 1328, multiple trend lines 1320 may be displayed when the detection unit in question (the ataxia detector 605 of FIG. 6) produces multiple likelihood measurements, such as for multiple limbs. In such a case, trend labels 1332, color coding, and/or a legend (not shown) may be provided to distinguish between the trend lines 1320.

The user interface 1302 provides the physician with a compact and readily understood view of the stroke likelihood data provided by the system 600 of FIG. 6, allowing the physician to observe the calculated likelihoods from each of the detectors over time and in response to various instructions, e.g., asking the patient to raise an arm and/or smile. All of the data may be correlated on a common time axis, allowing the physician to see at which point in a conversation with the patient certain events occurred, as well as trends for each of the stroke likelihood calculations. Moreover, the user interface 1302 provides confidence scores in the form of individual likelihoods 1308, threshold values 1310, and the like, as well as an overall assessment of whether the patient 1306 is experiencing (or has experienced) a stroke based on a combination of all of the factors. Finally, the user interface 1302 provides an augmented view of the patient, including, in one embodiment, superimposed points and lines indicating how certain stroke likelihood calculations are being performed.

In addition to being useful in a telehealth consultation, the user interface 1302 could also assist the physician in an in-person consultation when displayed on an augmented reality device. In such an embodiment, the user interface 1302 may display objective calculations to supplement the physician's observations, allowing the physician to focus on one indication of a stroke while the system 600 of FIG. 6 automatically processes all of the indications simultaneously. As such, the user interface 1302 may increase the accuracy of the physician's diagnoses.

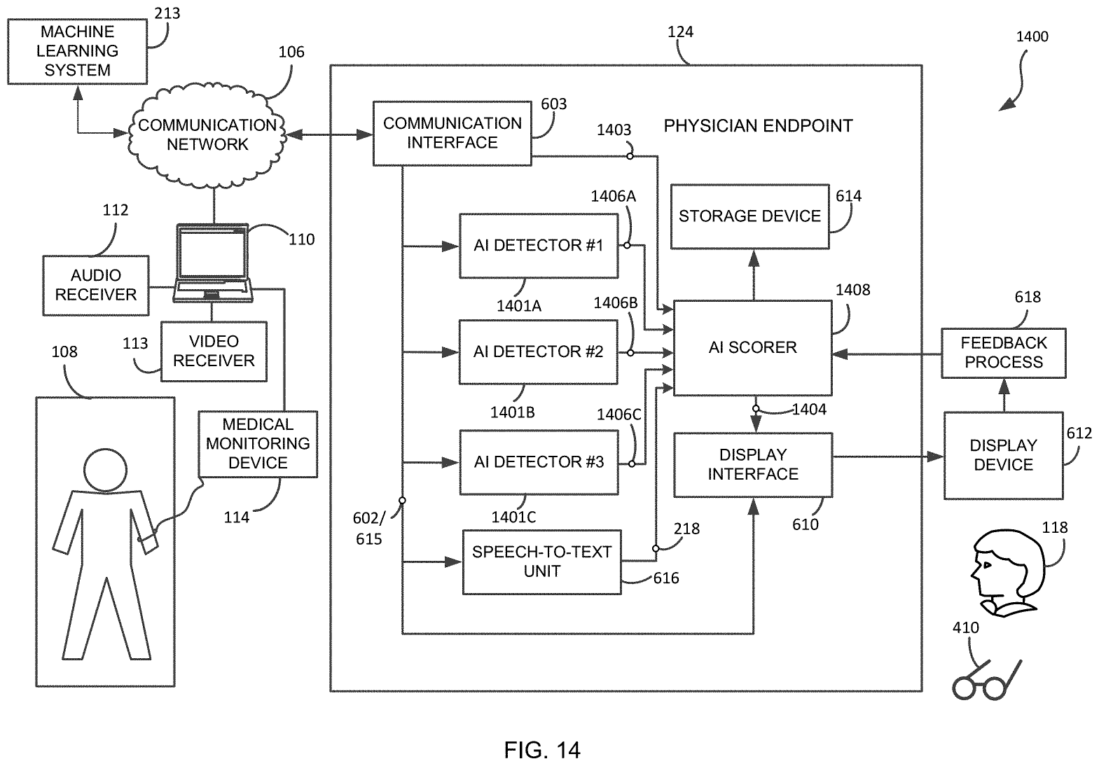

FIG. 14 illustrates a generalized system 1400 for automatically determining a health condition score based inputs from two or more different Al detectors 1401 (three depicted as 1401A-C). Examples of Al detectors 1401 may include the asymmetry detector 601, ataxia detector 605, and dysarthria detector 607 of FIG. 6, although other Al detectors 1401 may be used in different embodiments to diagnose health conditions besides stroke.

As previously noted, the Al detectors 1401 may receive one or both of an video stream 602 and an audio stream 615 from video receiver 113 (e.g., camera) and audio receiver 112 (e.g., microphone), respectively, in proximity to the patient 108. The video and audio streams 602, 615 may be received by the Al detectors 1401 through the communication network 106 via the communication interface 603. The Al detectors 1401 may be components of the physician endpoint 124 or accessed through communication network 106. For example, the Al detectors 1401 may make use of one or more machine learning systems 213 located remotely.

In addition to the Al detectors 1401, the system 1400 may include an Al scorer 1408, which is functionally similar to the stroke scorer 608 of FIG. 6 but adapted to different medical conditions. The system 1400 may also include a display interface 610, a display device 612, a storage device 614, and a speech-to-text unit 616, each of which may operate similarly to the components illustrated in FIG. 6.

Each Al detector 1401A-C may respectively process one or both of the audio and video streams 602, 615 using machine learning to automatically determine a respective likelihood 1406A-C of the patient 108 having a particular health condition. As discussed with reference to FIG. 6, the likelihoods 1406A-C may relate to whether the patient has experienced, or is experiencing, a stroke. In such an embodiment, the likelihoods 1406A-C may include a first stroke likelihood based on a measurement of facial droop, a second stroke likelihood based on a measurement of limb weakness, and a third stroke likelihood based on a measurement of slurred speech.

In other embodiments, only two Al detectors 1401 may be provided, generating two respective likelihoods 1406 of the health condition. In still other embodiments, four or more Al detectors 1401 may be provided, generating four or more respective likelihoods 1406 of the health condition.

In response to receiving the separate likelihoods 1406A-C from the Al detectors 1401A-C, the Al scorer 1408 generates an overall health condition score 1404, which may be similar to the stroke score 604 discussed with reference to FIG. 6. In calculating an overall health condition score 1404, the individual likelihoods 1406A-C may each be assigned a separate weight by the Al scorer 1408.

In one embodiment, the speech-to-text unit 216 converts the audio stream into text 218 that is combined by the Al scorer 1408 with the at least two likelihoods 1406 of the health condition using machine learning to automatically determine the overall health condition score 1404. The text 218 may be structured or unstructured and may distinguish between different voices, e.g., patient 108 and physician 118.

In certain embodiments, the Al scorer 1408 may be configured to receive diagnostic data 1403 from a medical monitoring device 114 in proximity to the patient. In such an embodiment, the Al scorer 1408 is configured to combine the diagnostic data 1403 with the at least two likelihoods 1401 of the health condition (and optionally the text 218) using machine learning to automatically determine the overall likelihood 1404 of the patient 108 having the health condition.

For example, the medical monitoring device 114 may comprise a heart rate monitor that provides cardiovascular measurements of the patient 108. Other types of diagnostic data 1403 may include, without limitation, electrocardiogram (ECG), Non-Invasive Blood Pressure (NIBP), temperature, respiration rate, and SpO2.

Beyond stroke, a variety of health conditions that may be evaluated by different Al detectors 1401 including, without limitation, mania, schizophrenia, aspirin poisoning, antihistamine poisoning, Parkinson's disease, amyotrophic lateral sclerosis (ALS), Bell's palsy, cerebral palsy, and multiple sclerosis (MS). Those skilled in the art will recognize that other conditions may be amenable to diagnosis by analyzing the video and/or audio streams 602, 615 using machine learning techniques.

Table 3 includes signals that are detectable through analyzing audio and video streams 602, 615 by different Al detection methods that are relevant to the likelihood that a patient 108 is suffering from mania, schizophrenia, aspirin poisoning, and antihistamine poisoning.

TABLE 3

| Signal | Description | AI Detection Method |
|---|---|---|
| Facial Action | Ekman's (EM) FACS | Convolutional Neural |
| Units | | Network (CNN) |
| Eye Gaze | Position of pupils relative | Eye tracking, CMU |
| Direction | to eyes + head orientation | Openpose |
| Head | Position of head relative | Pose estimator |
| Orientation | to a source, or the body | (OpenPose) |
| Cognitive | Limitations in mental | Question answering, |
| Delay | functioning and in skills, | facial analysis, pose |
| | such as communicating | estimation, speech |
| | | analysis |
| Pulse rate | Detect pulse rate during | Facial analysis |
| | sessionu sing video feed | |
| Respiratory | Detect respiratory rate during | Facial Analysis |
| rate | session using video feed | |
| Nystagmus | Repetitive, uncontrolled eye | Eye tracking |
| | movements | |
| Body | Track level of body activity | Pose estimation, body |
| activity | | landmarks |
| Face | Track level of face activity | Face landmark, head |
| activity | landmarks | pose |
| Face sentiment | Track sentiment in face | Facial analysis |
| Text sentiment | Sentiment in text during | Sound and text |
| | session | analysis |
| Rapid shift or | Sentiment tracking | Sentiment in video, |
| change in | | sentiment in text |
| behavior or | | |
| moods (cycling) | | |
| Voice stress | Level of stress in voice | Audio analysis |
| level | | |
| Dysarthria | Clarity/slur in speech | Dysarthria analysis |
| Speech content: | Rate of “I, me, my,” etc. | Speech analysis |
| personal- | usage | |
| pronoun use | | |
| Speech content: | Grammatical, syntactic | Speech analysis |
| completeness | completeness | |
| of sentences | | |
| Non-linguistic | Rates of “ur, um, uh,” etc. | Speech analysis |
| utterances | usage | |
| Pose-estimation/ | Creating a wire-frame of the | Pose estimator |
| body language | body from keypoints | |
| analysis | | |
| Temperature | Thermal imaging of patient | Video analysis |
| | skin | |
| Respiratory/ | Detect issues in lungs through | Audio analysis |
| wheeze | audio analysis | |
| detector | | |
| Rhinorrhea | Detection through image or | Audio/video |
| Detector | audio | analysis |
| Coloring in | Detecting level of red, yellow, | Color analysis |
| white of eye | etc. | |
| Blush/skin color | Level of redness/yellowness | Color analysis |
| change detection | in an area of skin vs normal | |
| Sweating | Excessive perspiration | Skin reflectivity |
| | | analysis |

Table 4 includes various audible and visual cues that are detectable by analyzing audio/video streams 602, 615 by different Al detectors 1401 for evaluating the likelihood of a patient 108 suffering from Parkinson's disease, amyotrophic lateral sclerosis (ALS), Bell's palsy, cerebral palsy, and multiple sclerosis (MS).

TABLE 4

| Condition | Audible cues | Visual cues |
|---|---|---|
| Parkinson's | Slurred speech | Tremor, slow movement |
| disease | | (bradykinesia), rigid muscles, impaired |
| | | posture and balance, loss of automatic |
| | | movements (blinking, swinging arms |
| | | while walking) |
| ALS | Slurred speech | Stumbling, difficulty holding items |
| | | with hands, poor posture, difficulty |
| | | holding head up, muscle stiffness |
| Bell's | Slurred speech | Drooling, inability to make facial |
| palsy | | expressions, facial weakness, facial |
| | | twitches, eye irritation (involved side) |
| Cerebral | Difficulty | Stiff muscles and exaggerated |
| palsy | speaking | reflexes (spasticity), stiff muscles with |
| | | normal reflexes (rigidity), lack of |
| | | balance (ataxia), tremors, slow/writing |
| | | movements, favoring one side of the |
| | | body, excessive drooling |
| MS | Slurred speech | Partial or complete loss of vision, |
| | | double vision, blurry vision |

After the overall health condition score 1404 is determined by the Al scorer 1408, it may be displayed to the physician 118 via the display interface 610 and/or stored in the storage device 614 with the text 218, diagnostic data 1403, video and/or audio streams 602, 615, and/or other data for subsequent review by the physician 118. In some embodiments, the physician 118 may be able to provide feedback via a feedback process 618 in order to update the models used by the Al scorer 1408.

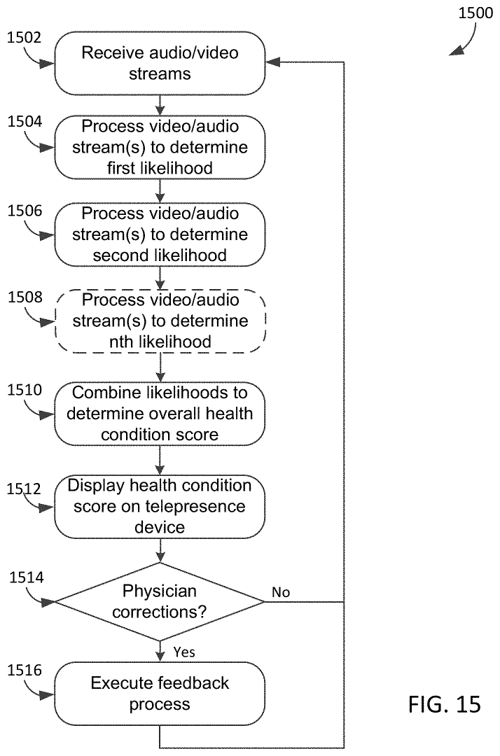

FIG. 15 is a flowchart of a method 1500 for automated health condition scoring based on a plurality of inputs generated by two or more Al detectors of the type shown in FIG. 14. Initially, audio and video streams are received 1502 from a telepresence device in a patient environment. The telepresence device could be a robotic endpoint, although the method 1500 is not limited in that respect. The video stream may include video frames that show a patient's face including, for example, the patient's eyes and lips, as well as one or more of the patient's limbs. The audio stream may include the patient's and/or physician's spoken voice.

In one embodiment, a video and/or audio stream is processed 1504 by a first Al detector using machine learning to automatically determine a first health condition likelihood. Concurrently or contemporaneously, the video and/or audio stream may also be processed 1506 by a second Al detector using machine learning to automatically determine a second health condition likelihood. Optionally, the video and/or audio stream may be processed 1508 by up to an nth Al detector to automatically determine an nth health condition likelihood. Each health condition likelihood may be independently determined using one or more machine learning systems, such as deep learning neural networks, using various input weightings and/or thresholds.

A health condition scorer combines 1510 the first, second, and up to nth health condition likelihoods to automatically determine an overall health condition score. The health condition score may be determined, in one embodiment, using a machine learning system, such as a deep learning neural network, which applies various weights and/or thresholds to the first, second, and up to nth likelihoods in calculating the overall health condition score.

Thereafter, an indication of the health condition score is displayed 1512 to a physician. The health condition score may be displayed, for example, with the video stream, audio stream, text generated from the audio stream, diagnostic data, and/or other information.

A determination 1514 is then made whether any physician corrections have been provided. If so, a feedback process 1516 is executed, by which one or more machine learning systems are updated or refined to incorporate the physician corrections. In either case, the method 1500 returns to continue receiving 1502 receiving the audio and video streams.

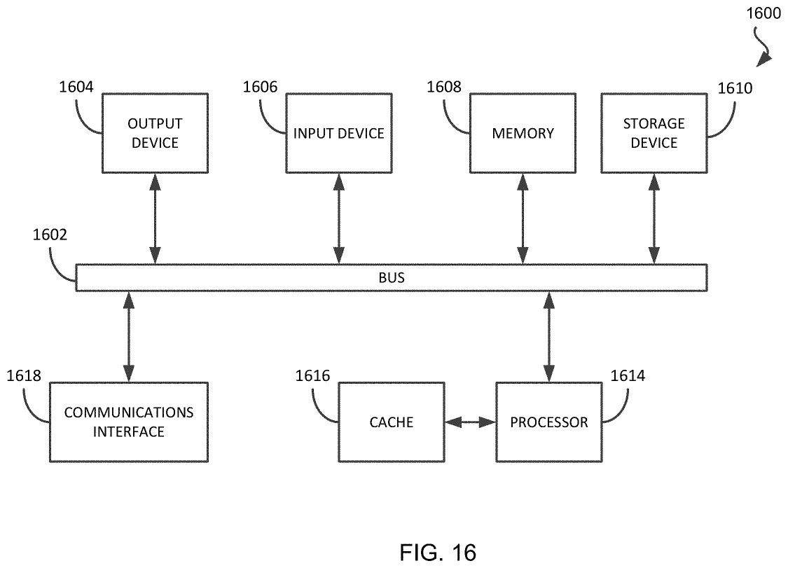

FIG. 16 depicts an example computer system 1600 that may implement various systems and methods discussed herein. The computer system 1600 includes one or more computing components in communication via a bus 1602. In one implementation, the computer system 1600 includes one or more processors 1614. Each processor 1614 may include one or more internal levels of cache 1616, as well as bus controller or bus interface unit to direct interaction with a bus 1602.

A memory 1608 may include one or more memory cards and control circuits (not depicted), or other forms of removable memory, and may store various software applications including computer executable instructions, that when run on the processor 1614, implement the methods and systems set out herein. Other forms of memory, such as a mass storage device 1610, may also be included and accessible, by the processor (or processors) 1614 via the bus 1602.

The computer system 1600 may further include a communications interface 1618 by way of which the computer system 1600 can connect to networks and receive data useful in executing the methods and system set out herein as well as transmitting information to other devices. The computer system 1600 may include an output device 1604, such as graphics card or other display interface by which information can be displayed on a computer monitor. The computer system 1600 can also include an input device 1606 by which information is input. Input device 1606 can be a mouse, keyboard, scanner, and/or other input devices as will be apparent to a person of ordinary skill in the art.

The system set forth in FIG. 14 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

1. A system for automated health condition scoring comprising:
at least one communication interface to receive an audio stream and a video stream from an endpoint in proximity to a patient;
at least two different artificial intelligence (“Al”) detectors to respectively process one or both of the audio stream and the video stream using machine learning to automatically determine at least two respective likelihoods of the patient having a health condition;
an Al scorer to combine the at least two respective likelihoods of the health condition using machine learning to automatically determine a health condition score representing an overall likelihood of the patient having the health condition; and
a display interface that displays an indication of the health condition score to a physician.

2. The system of claim 1, wherein the Al scorer assigns a separate weight to each of the at least two respective likelihoods of the health condition in determining the health condition score.

3. The system of claim 1, further comprising:
a speech-to-text unit to convert the audio stream into text that is combined by the Al scorer with the at least two respective likelihoods of the health condition using machine learning to automatically determine the overall likelihood of the patient having the health condition.

4. The system of claim 1, wherein the at least one communication interface receives diagnostic data from a medical monitoring device in proximity to the patient, and wherein the Al scorer is configured to combine the diagnostic data with the at least two respective likelihoods of the health condition using machine learning to automatically determine the overall likelihood of the patient having the health condition.

5. The system of claim 1, wherein the health condition is a stroke, and wherein the at least two different Al detectors are selected from a group consisting of an asymmetry detector, an ataxia detector, and a dysarthria detector.

6. The system of claim 1 wherein the health condition is a stroke, and wherein the at least two different Al detectors comprise three Al detectors including an asymmetry detector, an ataxia detector, and a dysarthria detector.

7. The system of claim 6, wherein:
the Al scorer comprises a stroke scorer;
the asymmetry detector processes the video stream to automatically determine a first stroke likelihood based on a measurement of facial droop;
the ataxia detector processes the video stream to automatically determine a second stroke likelihood based on a measurement of limb weakness;
the dysarthria detector processes the audio stream to automatically determine a third stroke likelihood based on a measurement of slurred speech; and
the stroke scorer automatically determines a stroke score for the patient based on a combination of the first, second, and third stroke likelihoods.

8. The system of claim 7, wherein the stroke scorer assigns a separate weight to each of the first, second, and third stroke likelihoods in calculating the stroke score.

9. The system of claim 8, wherein the stroke scorer assigns each separate weight using a machine learning system.

10. The system of claim 9, wherein the machine learning system comprises a deep learning neural network.

11. The system of claim 9, further comprising a feedback process to update the machine learning system based on physician feedback.

12. The system of claim 7, wherein the stroke score comprises at least one of a probability, a percentage chance or a confidence level of whether the patient has experienced, or is experiencing, a stroke.

13. The system of claim 7, wherein the stroke scorer compares the first, second, and third stroke likelihoods with respective thresholds in calculating the stroke score.

14. The system of claim 13, wherein the stroke score includes the first, second, and third stroke likelihoods and the respective thresholds.

15. The system of claim 13, wherein the stroke score includes a binary indication of whether or not the patient has experienced, or is experiencing, a stroke based on the respective thresholds.

16. The system of claim 7, wherein the video stream includes one or more video frames showing at least eyes and lips of the patient, and wherein the asymmetry detector comprises:
a facial landmark detector to automatically identify a set of facial keypoints in at least one of the one or more video frames, the facial keypoints including at least a point on each eye of the patient and at least one point on opposite sides of the patient's lips;
a facial droop detector in communication with the facial landmark detector to automatically calculate a degree of facial droop by calculating a first line between each eye point, calculating a second line between each lip point, and calculating an angle between the first line and the second line; and
an asymmetry scorer to automatically determine the first stroke likelihood based on the calculated angle.

17. The system of claim 16, wherein the facial landmark detector includes or makes use of a deep learning neural network in automatically identifying the set of facial keypoints.

18. The system of claim 16, wherein the facial droop detector comprises or accesses a deep learning neural network.

19. The system of claim 7, wherein the video stream includes one or more video frames showing a limb of the patient, and wherein the ataxia detector comprises:
a pose estimator to automatically identify body keypoints in the one or more video frames, the body keypoints including locations of joints on the limb of the patient,
a limb velocity detector to use the body keypoints to automatically determine a movement velocity of the limb over a time interval in which the patient is instructed to keep the limb motionless; and
a limb weakness scorer to automatically calculate the second stroke likelihood as a function of the movement velocity of the limb over the time interval.

20. The system of claim 19, wherein the limb velocity detector determines the movement velocity of the limb by calculating a sum of movement velocities for each joint of the limb.

21-61. (canceled)
